(19) United States
(12) Patent Application Publication
(10) Pub. No.: US 2026/0016494 A1
(43) Pub. Date: Jan. 15, 2026

(54) CARTRIDGES AND INSTRUMENTS FOR SAMPLE ANALYSIS

(71) Applicant: INTEGENX INC., Pleasonton, CA (US)

(72) Inventors: James Ogg, Sunnyvale, CA (US); David Eberhart, Santa Clara, CA (US); William D. Nielsen, San Jose, CA (US); Helen Franklin, San Jose, CA (US); Stevan B. Jovanovich, Livermore, CA (US)

(73) Assignee: INTEGENX, Pleasonton, CA (US)

(21) Appl. No.: 19/273,685

(22) Filed: Jul. 18, 2025

Related U.S. Application Data
(62) Division of application No. 16/258,412, filed on Jan. 25, 2019, now Pat. No. 10,989,723.
Application No. 16/258,412 is a division of application No. 15/037,039, filed on May 16, 2016, now Pat. No. 10,191,071.

(51) Int. Cl. G01N 35/00; B01L 3/00; B01L 7/00; C12Q 1/6818; C12Q 1/686; C12Q 1/6888

(52) U.S. Cl. CPC G01N 35/00663; B01L 3/502; B01L 3/502715; B01L 3/50273; B01L 3/502738; B01L 3/502746; B01L 7/52; C12Q 1/6818; C12Q 1/686; C12Q 1/6888; B01L 2200/026; B01L 2200/027; B01L 2200/04; B01L 2200/0689; B01L 2200/10; B01L 2200/16; B01L 2300/044; B01L 2300/0627; B01L 2300/0645; B01L 2300/0672; B01L 2300/0816; B01L 2300/0864; B01L 2300/0867; B01L 2300/087; B01L 2300/0887; B01L 2300/12; B01L 2300/123; B01L 2300/18; B01L 2300/1827; B01L 2400/0421; B01L 2400/0478; B01L 2400/0487; B01L 2400/0622; B01L 2400/0638; B01L 2400/0644; B01L 2400/0655; G01N 2035/00673

(57) ABSTRACT

A cartridge can include a first layer, a second layer and a deformable layer. The first layer comprises a first side contacting the deformable layer, the first side comprising a plurality of fluidic circuits each comprising at least one fluidic channel and at least one valve seat, a second side comprising at least one pneumatic distribution channel, at least one via in the first layer configured to put a pneumatic distribution channel in fluidic communication with the deformable layer. Portions of the deformable layer, with valve seats in the first layer, form diaphragm valves. The cartridge can include an actuation fluidic circuit comprising at least one branch channel and configured to actuate a diaphragm against a valve seat in a different fluidic circuit. Positive or negative pressure actuates a diaphragm portion of the deformable layer into or out of contact with the valve seat in the branched channel.

### CROSS-REFERENCE TO RELATED APPLICATIONS

This application is a continuation of U.S. application Ser. No. 17/211,212, filed Mar. 24, 2021, which is a divisional of U.S. application Ser. No. 16/258,412, filed Jan. 25, 2019 (now U.S. Pat. No. 10,989,723), which is a divisional of U.S. application Ser. No. 15/037,039, filed May 16, 2016 (now U.S. Pat. No. 10,191,071), which is a nationalization of PCT Application No. PCT/US2014/066008, filed Nov. 17, 2014 (now expired), which claims the benefit of and priority to U.S. Provisional Application No. 61/981,678 filed Apr. 18, 2014 (now expired) and U.S. Provisional Application No. 61/905,804, filed Nov. 18, 2013 (now expired), each of which is incorporated herein by reference in its entirely.

### STATEMENT AS TO FEDERALLY SPONSORED RESEARCH

None.

### BACKGROUND OF THE INVENTION

One barrier to the broad adoption of rapid DNA-based human identification is the consumable cost. A low-cost cartridge using small amounts of reagents would reduce this barrier. Previous approaches have focused on automation and manufacturing process improvement to reduce the cost to make a given cartridge.

Versions of systems including sample cartridges and fluidic systems for sample extraction and analysis are described in, for example, U.S. Pat. No. 6,190,616 (Jovanovich et al.); 6,551,839 (Jovanovich et al.); 6,870,185 (Jovanovich et al.); 7,244,961 (Jovanovich et al.); 7,445,926 (Mathies et al.); 7,799,553 (Mathies et al.); 8,173,417 (Tan et al.); 8,206,974 (Tan et al.); 8,394,642 (Jovanovich et al.); 8,425,861 (Selden et al.); 8,431,340 (Jovanovich et al.); 8,720,036 (Selden et al.) and 8,858,770 (Tan et al.); US patent applications 2009/0178934 (Jarvius); 2009/0253181; 2011/0039303 (Jovanovich et al.); 2011/0126911 (Kobrin et al.); 2011/0220502 (Selden et al.); 2012/0181460 (Eberhart et al.); 2013/0139895 (Vangbo) and 2013/0115607 (Nielsen et al.); and International Patent Application WO/US2013/130910.

### SUMMARY OF THE INVENTION

Fluidic devices are provided, for example in the form of a cartridge, for sample extraction and analyte reaction and analysis.

Provided herein is a cartridge comprising one or more fluidic circuits that each comprise at least one diaphragm valve; wherein the cartridge comprises: (a) a body comprising (i) a surface comprising a valve seat in fluidic communication with a valve inlet and a valve outlet and (ii) at least one port in fluidic communication with the fluidic circuit; and (b) a layer of deformable material covering the valve seat and the at least one port, wherein a portion of the layer of deformable material functions as a diaphragm which, in combination with the valve seat, forms a diaphragm valve, and wherein the diaphragm, when actuated (for example, by being moved into contact with the valve seat or by being moved out of contact with the valve seat), regulates fluid flow across the diaphragm valve, and comprising at least one conduit through the layer of deformable material, each conduit communicating with a port; and wherein the cartridge is configured to engage a cartridge interface, putting the deformable material in direct contact with a surface of cartridge interface, putting the diaphragm in communication with a source of positive and/or negative pressure that actuates the diaphragm (e.g., pneumatically or mechanically) and putting the at least one port in communication with a fluid or pneumatic line through the conduit, wherein the layer of deformable material optionally functions as a gasket sealing the at least one port against leakage. In one embodiment the fluidic circuit further comprises a reaction chamber formed in the body, optionally covered with a film of heat conductive material (e.g., a metal film). In another embodiment the fluidic circuit further comprises a chamber configured to receive a sample comprising a biological material, said chamber optionally comprising a close tab. An example of such an instrument and cartridge is shown in FIGS. 1-5.

Also provided herein is an instrument comprising: (a) at least one cartridge interface comprising: (i) an engagement unit configured to receive a removably insertable cartridge and to engage a received cartridge with a manifold assembly and, optionally, a thermal regulator, wherein the cartridge comprises at least one fluidic circuit comprising at least one fluid channel, at least one exit port; and at least one diaphragm valve comprising a valve seat configured to regulate fluid flow in the at least one fluidic circuit; and a layer of deformable material covering the valve seat and the at least one port, wherein the deformable material functions as a diaphragm in the diaphragm valve and comprises at least one conduit through the layer, each conduit communicating with a port; and (ii) a manifold assembly comprising an engagement surface configured to directly contact the deformable layer of a received cartridge and having a plurality of transmission channels communicating with ports on the engagement surface; wherein engaging a cartridge with the manifold assembly: (A) puts the diaphragm in communication with a first port on the engagement surface configured to transmit positive or negative pressure to the diaphragm, and (B) puts the exit ports in communication with second ports on the engagement surface configured to transmit fluid into or out of a fluidic circuit through the transmission channels; and (iii) a thermal regulator which, when engaged with a received cartridge, puts a heat pump (e.g., a thermoelectric heater, e.g., a Peltier device) in thermal contact with a thermal cycling chamber in the cartridge or a heating element in thermal contact with a chamber in the cartridge. In one embodiment the instrument of further comprises any of: (b) a pneumatic pressure source configured to deliver positive or negative pneumatic pressure to a transmission channel of the manifold assembly; (c) a pump configured to move liquid into or out of a transmission channel of the manifold assembly; (d) a source of reagents in fluid communication with a transmission channel of the manifold assembly; (e) an analysis module configured to perform at least one analysis (e.g., electrophoresis) on a fluid received from the cartridge; and (f) a control module comprising executable code that, when executed, controls operation of the instrument.

Also provided herein is a cartridge comprising a first layer and a deformable layer: (a) wherein the first layer comprises: (i) a first side contacting the deformable layer, wherein the first side comprises a plurality of fluidic circuits, each fluidic circuit comprising at least one fluidic channel; and (ii) a second side comprising at least one fluidic distribution channel, which fluidic distribution channel is covered by a cover layer; (iii) a plurality of vias in the first layer, each via configured to put the fluidic distribution channel in communication with a fluidic channel; and (iv) optionally, at least one via in the first layer configured to put the fluidic distribution channel in communication with a port on a side of the first layer configured to engage a source of fluid; and (b) optionally comprising an actuation layer comprising at least one actuation circuit configured to actuate a diaphragm against a valve seat in the first side. An example of such a cartridge is shown in FIGS. 6 and 9.

Also provided herein is a cartridge comprising a first layer, a second layer and a deformable layer sandwiched there between: (a) wherein the first layer comprises: (i) a first side contacting the deformable layer, wherein the first side comprises a plurality of fluidic circuits, each fluidic circuit comprising at least one fluidic channel and at least one valve seat; (ii) a second side comprising at least one pneumatic distribution channel, which pneumatic distribution channel is optionally covered by a cover layer; (iii) at least one via in the first layer configured to put a pneumatic distribution channel in communication with the deformable layer; and (iv) optionally, at least one via in the first layer configured to put a pneumatic distribution channel in communication with a port on a side of the first layer configured to engage in a source of pneumatic pressure; (b) wherein portions of the deformable layer, in combination with valve seats in the first layer form diaphragm valves; and (c) wherein the actuation layer comprises at least one actuation circuit comprising at least one branch channel, wherein each branch channel is configured to actuate a diaphragm against a valve seat in a different fluidic circuit and further comprises a valve seat; and wherein positive or negative pressure applied to a pneumatic distribution channel transmits pressure through the via to actuate a diaphragm portion of the deformable layer into or out of contact with the valve seat in the branched channel, wherein closing the control valve inhibits actuation the diaphragm valve in the fluidic circuit. An example of such a configuration is shown in FIG. 10A-B.

Also provided herein is a cartridge comprising: (a) a body comprising a polymer and comprising at least one functional feature on a surface of the body and configured to transmit fluid (e.g., a port, a via, a fluid channel, a chamber, a valve inlet and valve outlet and/or a valve seat); and (b) a layer of deformable material thermally bonded to the body and covering the functional feature (optionally, wherein at least one portion of the layer comprises a permanent deformation), and wherein application of positive or negative pressure to at least one portion of the layer actuates the deformable material into or out of contact with a functional feature on the surface of the body; and wherein the cartridge is configured to engage a cartridge interface configured to supply positive or negative pressure to the at least one portion of the layer. In one embodiment the cartridge further comprises a fluid-filled chamber in the body, wherein the chamber has an opening sealed closed with a heat seal material. In another embodiment the deformable material comprises a heat seal material. In another embodiment the deformable material comprises a material selected from polypropylene, polyethylene, polystyrene, cycloolefin co-polymer (COC), mylar, polyacetate and a metal. An example of valves in such a cartridge is shown in FIGS. 7, 8, 13, and 14 (seal not shown in all figures).

Also provided herein is a device comprising the aforementioned cartridge and a ram configured to actuate a diaphragm of a diaphragm valve on the body.

Also provided herein is a device comprising a cartridge and a clamping device: (a) wherein the cartridge comprises: (i) a body comprising: (A) at least one fluidic circuit comprising: (I) at least one functional feature on a surface of the body and configured to transmit fluid (e.g., a port, a fluid channel, a chamber, a valve inlet and valve outlet and/or a valve seat), wherein the functional feature optionally comprises a ridge on the surface of the body; and (II) at least one compartment containing a liquid, wherein the compartment communicates with the functional feature through one or more vias in the body; and (ii) a layer of deformable material covering the functional feature; and (b) wherein the clamping device, when engaged with the cartridge, applies sufficient pressure to the deformable material to deform the deformable material against the cartridge body and seal against movement of liquid from the compartment and through the at least one functional feature; and wherein removing the clamping device releases pressure to the deformable material, allowing the seal to open (e.g., through an elastic response of the deformable material or through application of positive or negative pressure against the seal). In one embodiment the clamping device comprises a mechanical clamp or a vacuum seal. An example of such a cartridge is shown in FIGS. 6 and 11.

Also provided herein is a cartridge comprising: (a) a fluidics layer comprising a surface having at least one diaphragm valve comprising a valve seat (e.g., a recessed valve seat); (b) a deformable layer mated to the surface, wherein a portion of the deformable layer functions as a diaphragm which, when actuated, is configured to move into or out of contact with the valve seat; and wherein the portion of the deformable layer functioning as a diaphragm comprises a boss positioned on a side of the deformable layer opposite of a side that contacts the valve seat; and (c) optionally comprising: a rigid substrate mated with the deformable layer and comprising apertures exposing the bosses and configured to receive a ram that contacts the boss and actuates the diaphragm, e.g., by application of mechanical pressure; or configured to engage an interface comprising apertures exposing the bosses and configured to receive a ram that contacts the boss and actuates the diaphragm, e.g., by application of mechanical pressure. An example of such a cartridge is shown in FIGS. 15A and B.

Also provided herein is a cartridge comprising: (a) a base comprising: (I) a central barrel comprising a pump chamber and movable syringe, (II) a base floor comprising a port station comprising a floor port; and (III) a channel fluidically connecting the barrel chamber to the port in the floor; and (b) a turret configured to revolve around the central barrel and comprising a plurality of turret chambers, each turret chamber comprising a turret chamber aperture in a chamber floor of the turret chamber and facing the base floor, wherein positioning a turret chamber at the port station puts the turret chamber aperture in fluid communication with the barrel chamber through the floor port, and wherein the floor closes a turret chamber aperture when the turret chamber is positioned at at least one position other than the port station; and wherein at least one turret chamber further comprises a channel communicating between the floor port and an exit port. An example of such a cartridge is shown in FIG. 17.

Also provided herein is an instrument comprising a cartridge interface and a removable cartridge engaged therewith: (a) wherein the interface comprises a base and one or more hollow bore pins for delivering fluid to a port in the cartridge, wherein the pin is biased against the base by the cartridge and protrudes through an aperture in the base; and wherein the pin comprises a home lead-in configured to put the pin in a home position after the bias is released by removal of the cartridge; and a tapered end configured to mate with a guide in the cartridge; and (b) wherein the cartridge comprises a fluidic circuit comprising a port and a pin guide communicating with the port, wherein the pin guide comprises a mating cone configured to mate with a tapered end of the pin and a pin guide lead-in configured to guide the pin into the mating cone when the cartridge exerts a bias against the tapered end of the pin during engagement with the interface. An example of such a combination is shown in FIG. 18.

Also provided herein is a cartridge comprising: (a) a body comprising at least one puncturing element, at least one fluidic channel and at least one flange; and (b) a reagent reservoir comprising at least one fluidically isolated, fluid filled compartment; a breakable seal in a wall of the compartment and a breakable tab attached to an outside wall of the compartment; wherein the cartridge is configured such that when the reagent reservoir is engaged with the body: (i) the puncturing elements punctures the breakable seal, putting the compartment in fluidic communication with the channel; and (ii) the flange exerts a force against the breakable tab, breaking the tab and forming a vent in the compartment. An example of such an embodiment is shown in FIG. 12.

Also provided herein is a fluidic device comprising one or more diaphragm valves, each diaphragm valve configured to regulate fluid flow in a fluidic channel, wherein the fluidic device comprises a fluidics layer, an actuation element and a deformable membrane, wherein each diaphragm valve comprises: a) a diaphragm comprised in the deformable membrane; b) a valve seat comprised in the fluidics layer and recessed from a surface of the fluidics layer that contacts the deformable membrane so that the diaphragm does not close the diaphragm valve unless positive pressure is exerted on the diaphragm; and c) a valve inlet and a valve outlet comprised in the fluidics layer and in fluid communication with a fluidic channel; and d) a ram configured to actuate the diaphragm; wherein the deformable membrane comprises a plastic material adhered to the fluidics layer through a heat activated adhesive, thermal fusion, chemical bonding or a pressure sensitive adhesive, and wherein the deformable membrane is configured such that pressure on the deformable membrane presses the membrane against the valve seat, thereby closing the valve and wherein the valve can be opened by releasing pressure of the ram against the deformable membrane or by pushing liquid through the valve. In one embodiment the deformable membrane is not an elastomeric material, e.g., is not PDMS. In another embodiment the deformable membrane comprises a laminate comprising the plastic material and a deformable, space-filling material, wherein pressure against the space-filling material causes the space filling material to fill the valve chamber sufficiently so that of the plastic material closes the valve. In another embodiment the deformable material has a durometer value of between 10 to 80 Shore D. In another embodiment the deformable material has a thickness sufficient such that the mechanical pressure is applied the deformable material sufficiently fills the valve chamber to form a seal between the plastic material in a valve seat to close the valve. In another embodiment the deformable, space-filling material comprises a solid foam. In another embodiment the deformable material is attached to the plastic material through an adhesive. In another embodiment the deformable material is pressed into contact with the plastic layer through an interface device. In another embodiment the fluidics layer comprises a polymer, e.g., a thermoplastic.

Also provided herein is a fluidic device comprising one or more diaphragm valves, each diaphragm valve configured to regulate fluid flow one or more fluidic channels, wherein the fluidic device comprises a fluidics layer, an actuation element and a deformable membrane, wherein each diaphragm valve comprises: a) a diaphragm comprised in the deformable membrane; b) a valve seat comprised in the fluidics layer and recessed from a surface of the fluidics layer that contacts the deformable membrane so that the diaphragm does not close the diaphragm valve unless positive pressure is exerted on the diaphragm; and c) a valve inlet and a valve outlet comprised in the fluidics layer and in fluid communication with a fluidic channel; and d) a ram comprised as an actuation element having a forked end comprising tines, wherein the end has a surface complying with the valve seat and wherein the tines are compliant to lateral pressure whereby pressure by the ram on the diaphragm and against the valve seat closes the valve. Embodiments are shown in FIGS. 29-32.

Also provided herein is a fluidic device comprising a sample input, a sample output and a waste chamber, all fluidically connected through fluid channels wherein the waste chamber comprises a material that degrades nucleic acid. In one embodiment the material that degrades nucleic acid comprises a hypochlorite salt. In another embodiment the material that degrades nucleic acid comprises an enzyme such as an exonuclease or an endonuclease.

Also provided herein is a fluidic device comprising a fluidic circuit comprising sample input, a reaction chamber and a sample output, wherein the reaction chamber comprises a solid substrate, e.g., solid phase extraction material, for retaining analyte from a sample. In one embodiment the solid substrate comprises a material that binds nucleic acid. In another embodiment the solid substrate comprises Whatman FTA paper, a carboxylated material, a sponge-like material, a polymer membrane, or glass particles. In another embodiment the solid substrate binds a predetermined amount of material. Embodiments are shown in FIGS. 22-28.

Also provided herein is a method comprising 1. A method comprising: (a) providing a reaction mixture comprising: (I) a sample comprising mammalian (e.g., human) DNA, (II) reagents for amplifying short tandem repeats (STRs) in the mammalian DNA (e.g., labeled primers, nucleotides and polymerase) and (III) a mammalian-specific probe selected to be amplified in the reaction and including a label that is distinguishable from the labeled primers; (b) performing an STR reaction comprising amplifying STRs in the sample and the mammalian specific probe; (c) detecting an amount of amplified mammalian specific probe in the reaction, e.g., over time, e.g., performing real-time PCR; and (d) optionally, stopping the STR reaction based on the amount of amplified mammalian specific probe detected. In one embodiment the labels are fluorescent labels and the distinguishable label has a wavelength above or below the highest or lowest wavelength of labeled primers. In another embodiment the mammalian specific probe further comprises a quencher such as a Black Hole Quencher® or a TaqMan® probe.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative claims, in which the principles of the disclosure are utilized, and the accompanying drawings of which:

FIG. 1 shows an instrument 101 comprising an interface adapted to engage a cartridge, including a cartridge interface 103 and a cartridge 200 inserted into a slot 107. The embodiment shown contains four cartridge-receiving subassemblies and Peltier device 109.

FIG. 2 shows cartridge 200, which is insertable into instrument 101. The cartridge includes a body 201. It further includes elastic layer 203 attached to a surface of the body. Layer 203 provides a diaphragm for a diaphragm valve on a surface of body 201, as well as ports 215 communicating with fluidic channels in body 201. The cartridge also includes cover sheet 205 that seals a chamber in the body and/or functions to transmit heat to or from the chamber. For example, when the cartridge is engaged with the interface, the chamber can function as a thermal cycling chamber and cover sheet 205 can be in contact with a source of thermal energy, such as a Peltier device 109. The cartridge also includes a slot 207 adapted for receiving a sample. Aperture 209 and notch 225 are alignment features configured to align cartridge 200 with cartridge interface 103. The cartridge also includes a closable cap 211.

FIG. 3 shows a cutaway view of cartridge 200. Reaction chamber 301 is in thermal contact with the cover sheet 205 which can be sealed to the body. Cartridge 200 includes diaphragm valve 303. Diaphragm valve 303 includes recessed valve seat 305 defining a valve chamber. Valve inlet 306 and valve outlet 307 are configured as vias through body 201 and communicate with fluidic channels in the body. Deformable layer 203 includes a portion functioning as a diaphragm 311. Putting diaphragm 311 into contact with valve seat 305 closes diaphragm valve 303. Deformable layer 203 also comprises port 309 that communicates with a fluidic conduit in the cartridge. When the cartridge is engaged with a cartridge interface, deformable layer 203 functions as a gasket that seals around port 309.

FIG. 4 shows from one aspect an exploded view of a cartridge 200 and an assembly 401 of a cartridge interface. The cartridge includes body 201, cover sheet 205, and deformable layer 203. The body includes, on one side, fluidic channel 213 and reaction chamber 301. Fluidic channels communicate through apertures in body 201 with a face covered by deformable layer 203. The deformable layer 203 includes ports 215 configured to align with ports 403 on the interface assembly 401 and an area 311 positioned to function as a diaphragm in a diaphragm valve. Fluidic lines 405 connect to interface assembly 401 and transmit fluids to port 403 which connects to cartridge 201. Pneumatic line 407 also connects to interface assembly 401 and transmits positive or negative pneumatic pressure to port 409 which actuates diaphragm for 311.

FIG. 5 shows from another aspect an exploded view of a cartridge of this disclosure and an assembly 401 of a cartridge interface. Body 201 includes a valve body with valve seat 305 with apertures 306 and 307 which is covered by deformable layer 203. Interface assembly (401) includes pneumatic line 407 that, when engaged with deformable layer 203, transmits positive or negative pressure to actuate diaphragm 311. Interface assembly 401 also includes fluid lines 405 communicating through passages in interface 401 with apertures 215 in the deformable layer 203 to allow movement of fluids into, through an out of fluidic circuits in the cartridge.

FIG. 6 shows cartridge 601 comprising a plurality of sample receptacles 607 and comprising reagent chambers in piece 905, distribution channels 903 that distribute reagents from reservoirs 603 across a plurality of fluidic circuits. Piece 905 includes reagent chambers 1107. Piece 905 is covered with deformable layer 907. Clamping elements 1103 apply pressure to deformable layer 907. Deformation of the deformable layer against piece 907 blocks movement of liquid through passages. This prevents movement of reagents through fluidic circuits during shipping.

FIG. 7 shows a cartridge 701 comprising a body 1205 having, on side 1311, fluidic channel 1315 and valve seat 1317. On another side, the cartridge has a reagent container having open compartments 1201 that can comprise a seal of a layer of material, for example through a heat seal, to fluidically isolate fluids contained in the compartments until use.

FIG. 8 shows a cartridge 801 comprising a body having a first side having open compartments 811, ports 813 and reaction chamber 815. The body also has a second side comprising valve seats 817. The layer 819 can be bonded to the first side of the body, sealing off the open compartments. The film 821 is capable of transmitting heat and will cover and seal reaction chamber 815. The deformable layer 1303 provides areas that function as diaphragms. When pressed against the second side, for example by a clamping device, the deformable layer 1303 is pressed into the valve seats, closing the valves and preventing fluid movement through fluidic circuits until use.

FIG. 9 shows an embodiment of a cartridge having a fluid distribution channel. Body 905 comprises a surface on one side that comprises a plurality of fluidic channels 901 oriented in a first direction. Body 905 also comprises a surface on a second side comprising a channel 903 having an orientation that is oblique to the first direction, for example, at right angles to the first direction. Channel 903 on a second side communicates with each of a plurality of fluidic channels on the first side through vias 904 that traverse body 905. Channel 903 is closed by a cover layer 907. Channel 903 optionally communicates with a source of fluid through a bore 920 in piece 905. Fluid channels 901 are covered by a deformable layer 909. Channels 901 also can comprise valve seats. Portions of the deformable layer can function as diaphragms to open or close the valves. These can be operated through pneumatic layer 911 comprising pneumatic channels that actuate the diaphragms. Alternatively, the cartridge can be engaged with an interface that provides an actuation force to the diaphragms.

FIGS. 10A and 10B show a segment of cartridge 1000 having a control line configured to disable a selected diaphragm valve in the cartridge. FIG. 10A shows an exploded view of the cartridge. FIG. 10 B shows an exploded view in clamshell format. The cartridge comprises a fluidics layer 1001, pneumatic layer 1003 and a deformable layer 1005 sandwiched between them. Fluidics layer 1001 includes at least one fluidic channel 1007 that includes recessed valve seat 1009. The fluidics layer also comprises a control line 1011 having a branch 1013 that communicates through a via 1015 with a surface of the fluidics layer mated with the deformable layer. The pneumatic layer comprises a pneumatic channel 1017 comprising one or more branches 1019. Each branch controls one diaphragm valve. The branch includes a valve relief 1021 positioned on the deformable layer opposite the valve seat in the fluidics layer into which the deformable layer can be deformed and which can transmit pressure to the diaphragm, actuating the diaphragm into the valve seat 1009. The branch also comprises a valve seat 1023 positioned opposite via 1015, which via connects to pneumatic control line 1011. Assertion of positive or negative pressure against control line 1011 to the diaphragm actuates the diaphragm against or away from the valve seat 1023 in the pneumatic layer. When pressed against the valve seat, this prevents transmission of pressure through pneumatic actuation channel 1017, rendering the diaphragm valve 1009 that controls fluid in fluidic line 1007 inoperative.

FIG. 11 shows a shipping clamp 1103 on cartridge 905.

FIG. 12 shows a cartridge comprising a body 1205 and a reagent reservoir 1201. The reagent reservoir has compartments, 1209, that can contain a liquid and that can be sealed with a layer such as heat seal, chemical seal, adhesive or laser weld. The body can comprise puncturing elements (not shown) to puncture seals in a floor of compartment 1209 when the reagent reservoir is pressed against the body. The puncturing elements can be a solid material, e.g., that protrude from the body, and that may have a tapered end that is pointed or sharp and that is adapted to apply concentrated pressure to the floor and to puncture a hole in the floor. This creates a fluidic connection between the compartment and a fluidic circuit in body 1205. This combination also includes flanges 1207 and breakable tabs 1203. When reagent reservoir 1201 is pressed against body 1205, flange 1207 engages tab 1203 and applies a force which breaks tab 1203, thereby creating a vent in compartment 1209. This allows fluid in compartment 1209 to drain through the aperture in the floor of the compartment and into the fluidic circuit.

FIG. 13 shows a diaphragm valve with a thin sealing layer 1303 that can comprise a heat seal material.

FIG. 14 shows a tool for introducing a dimple into a diaphragm.

FIGS. 15A and 15B show cartridge 1501 having a body 1503 attached to an deformable layer 1505 and having a cover 1507 on the deformable layer so that the deformable layer in the cartridge is not open-faced. This cartridge includes a diaphragm valve 1513 that is normally open and that regulates fluid flow along a fluidic channel 1515. The cover 1507 covers the deformable layer and comprises an aperture 1509. The deformable layer includes a boss 1511 fitted with the aperture. A mechanical actuator, such as a piston, can be used to close the valve by actuating the diaphragm by the provided boss. This cartridge further comprises a chamber 1517 for receiving a swab or other sample and a reaction chamber 1519 in fluidic communication with channel 1515. The reaction chamber 1521 can be covered with a seal and/or can have a separate heat spreader layer 1521.

FIGS. 16A and 16B show, respectively, a front and back view of a multi-sample cartridge.

FIG. 17 shows a revolving multi-chamber turret. Cartridge 1701 comprises a base 1703. The base has a central barrel 1705 comprising a pump chamber 1707 and movable syringe 1709, a floor 1711 comprising a port station comprising a port 1713 and a channel 1715 fluidically connecting the barrel chamber to the port in the floor. The cartridge also has a turret 1717 configured to revolve around the central barrel 1705 and comprising a plurality of turret chambers (e.g., 1719), each turret chamber comprising a turret chamber aperture 1721, wherein the turret is configured to rotate around the syringe barrel, wherein positioning a turret chamber at the port station puts the turret chamber aperture in fluid communication with the barrel chamber through the floor aperture and wherein the floor covers a turret chamber aperture when turret chamber is positioned at least one position other than port station. At least one turret chamber further comprises a channel communicating between the port and an exit port 1723.

FIGS. 18A and 18B shows cross-sectional views of a self-aligning, self-resetting pogo pin 1801 reset into its home position, and fluidically connected to the cartridge, respectively.

FIG. 19 shows an exploded view of an interface slot.

FIG. 20 shows a cartridge interface 1901.

FIG. 21 shows a schematic of a cartridge of this disclosure.

FIG. 22 shows a bottom view of a cartridge circuit.

FIG. 23 shows the valve states and flow for a cell lysis operation.

FIG. 24 shows the valve states and flow for nucleic acid capture.

FIG. 25 shows the valve states and flow for movement of liquid into a waste chamber.

FIG. 26 shows the valve states and flow for creation of a reaction mixture.

FIG. 27 shows the valve states for thermal cycling.

FIG. 28 shows the valve states and flow for movement of the amplification product to output ports.

FIG. 29 shows a cutaway view of a normally open valve actuated by a ram having a tip with flexible elements.

FIG. 30 shows a three-dimensional view of a valve of this disclosure.

FIG. 31 shows a valve of this disclosure in a closed configuration. A flexible end of the ram presses a deformable membrane against a valve seat. The ram is configured to press the deformable membrane so as to seal the valve inlet and the valve outlet by pressure against the perimeter of the inlet and outlet in the valve seat.

FIG. 32 shows a valve having a valve chamber defined by a recess in a fluidic layer and a diaphragm comprised in a laminate layer.

### DETAILED DESCRIPTION OF THE INVENTION

### I. Instruments for Engaging and Operating fluidic Cartridges

In one aspect provided herein is a cartridge comprising: (a) a body comprising a fluidic circuit comprising: (1) a sample chamber comprising an opening configured to receive a sample and a via through the body; (2) a reaction chamber; (3) diaphragm valve elements including a valve inlet and a valve outlet, each configured as a via through the body, and a valve seat; (4) a plurality of ports through the body; and (5) one or more fluidic channels in a surface of the body, wherein the one or more fluidic channels put the sample chamber (e.g., through the via), the reaction chamber, the valve elements (e.g., through the valve inlet and valve outlet), and each of the plurality of ports in fluidic communication with each other; (b) a cover layer attached to the body and sealing the via, the fluidic channels and the reaction chamber; and (c) a deformable layer attached to the body, wherein the deformable layer (i) comprises a plurality of holes, each hole communicating with a port; and (ii) in combination with the valve inlet, the valve outlet and the valve seat, form a diaphragm valve configured to regulate fluid flow in the fluidic circuit.

In another aspect provided herein is an instrument comprising: (a) at least one cartridge interface comprising: (1) an engagement assembly having a first position to receive a cartridge as described herein and a second position to engage a received cartridge with a manifold assembly and an optional thermal regulator; (2) a manifold assembly having a plurality of channels, each channel opening onto a front port and a back port, wherein, when the engagement assembly has received a cartridge and is in the second position, a plurality of front ports fluidically engage ports in the cartridge and pneumatically engage the diaphragm of the diaphragm valve, and wherein the deformable material serves as a gasket for fluidic engagement; (3) an optional thermal control assembly (e.g., thermal cycler) configured to place a heat spreader in thermal contact with a thermal cycling chamber of an engaged cartridge and to regulate temperature of the thermal cycling chamber, when the engagement assembly is the second position; (b) a pneumatic and fluidic assembly comprising: (1) a source of pneumatic pressure; (2) a plurality of fluid sources; (3) a plurality of transmission lines connecting a source of pneumatic pressure in each of the plurality of sources in fluid communication with a back port of the manifold assembly; (4) a pump configured to move fluids from the source through the transmission lines.

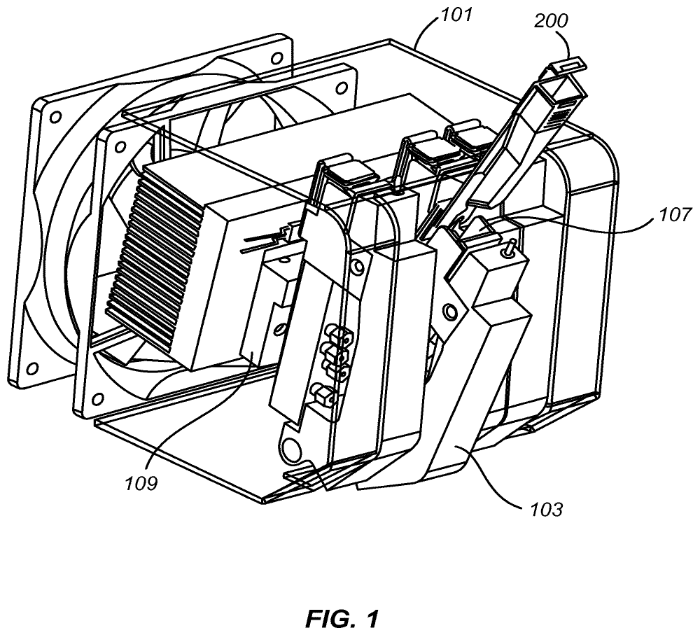

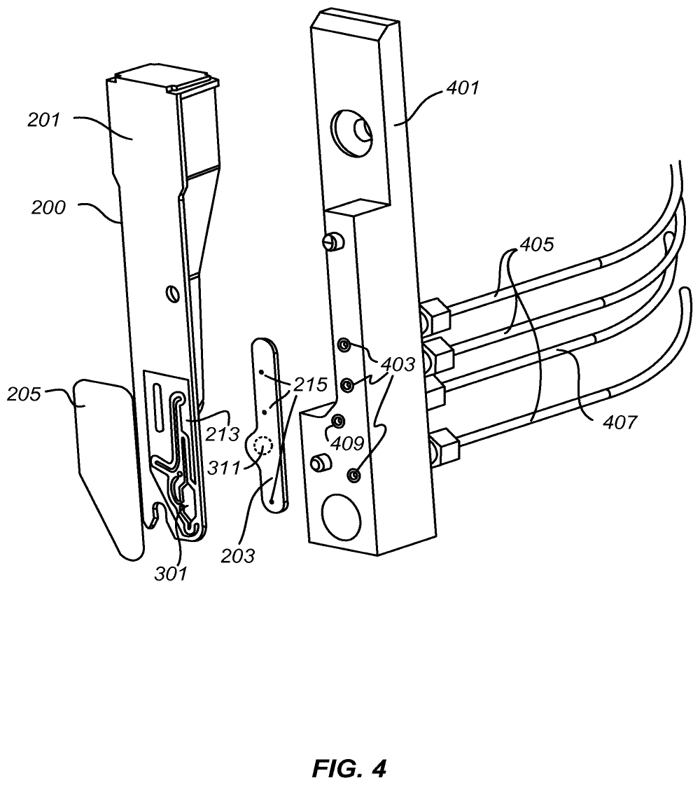

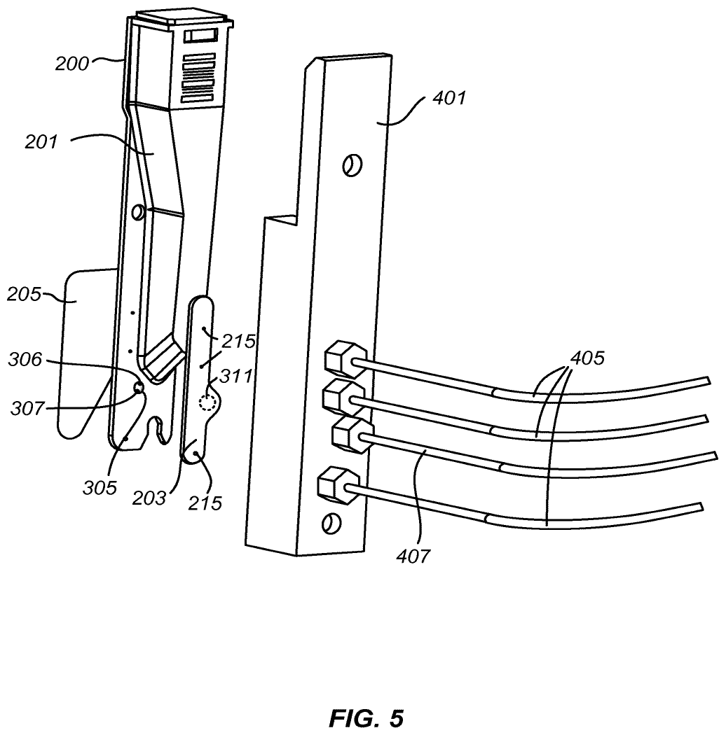

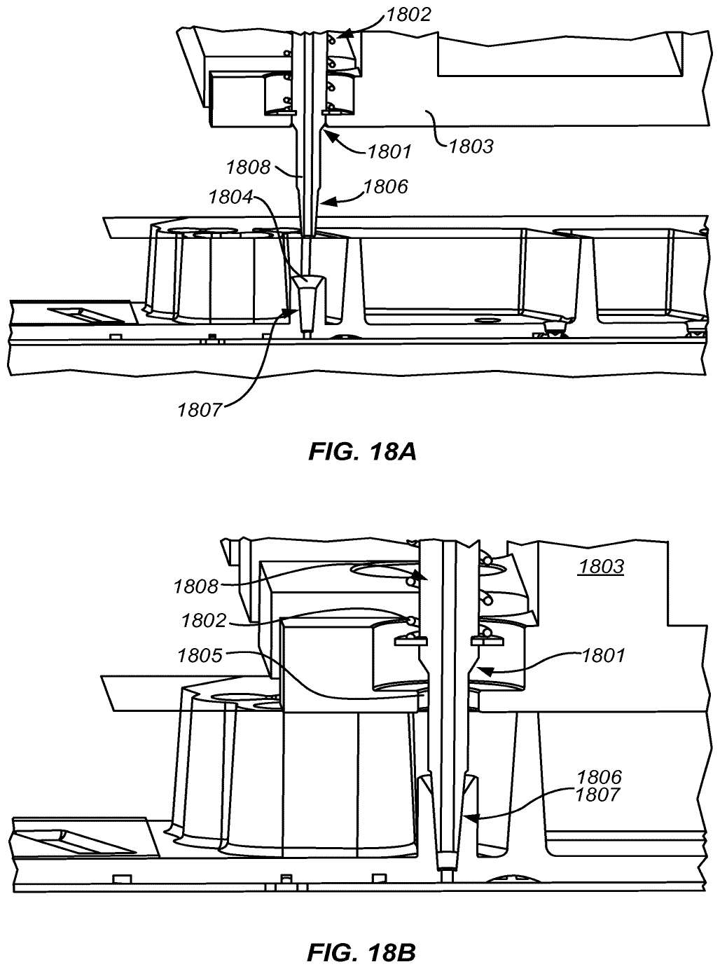

FIG. 1 shows an interface configured for a cartridge. Referring to FIGS. 4 and 5, for a single sample cartridge, the interface may also include three fluidic connections 405 and one pneumatic connection 407 to control the valve. These can be low-dead-volume connections which connect to the pneumatic and fluidic assembly through tubes. Alternatively, they can be rams such as pogo pins (e.g., 1801 of FIG. 18).

This configuration permits samples to be inserted into cartridges, and cartridges to be independently inserted into a slot, even if other slots are processing other samples. Thus, in one embodiment, the system can process samples independently.

The cartridge described in FIGS. 1-5 minimizes the cost of manufacture by minimizing the functions that need to be handled by the disposable cartridge. These functions are moved onto a pneumatic and fluidic assembly, which can be a permanent or semi-permanent part of the system.

In this embodiment, the cartridge can comprise an injection molded body, for example, a plastic, a deformable film; and a foil, such as a metal foil, each bonded to the body. The body can have integrated alignment features 209 and 225 so that it can be easily and accurately inserted into the interface. The plastic material can include any plastic known to those skilled in the art, such as polypropylene, polystyrene, polyethylene, polyethylene terephthalate, polyester, polyamide, poly (vinylchloride), polycarbonate, polyurethane, polyvinyldiene chloride, cyclic olefin copolymer (COC), or any combination thereof.

The cartridge may be scribed with a barcode or QR code for optical identification or have an EEPROM or RFID or other similar identification device mounted on the cartridge that can assist in sample tracking and optionally contain information about the chemistry, process to be performed, lot number, expiration date, and other information.

The body can have a folding tab 211 that can be snapped shut after the swab is inserted, either by the operator or the system. More than one style of body, each adapted to a swab, punch type, or sample type can be produced. After the tab is snapped shut, the body can serve to contain the sample, providing protection against contamination and facilitating re-testing or recovery of the sample as required.

The body can also define the volumes for two process chambers. The swab, punch, or other sample type is placed in a compartment 207 that also serves as a lysis chamber. To accommodate the swab, punch, or other sample type, it can have a volume ranging from, e.g., 10 μL to 15 ml or 1 ml to 10 ml. Cells are lysed and DNA extracted from the swab, punch, or other sample type in this chamber. The second chamber 301, called the reaction chamber, can serve to capture DNA or house a small amount of lysate for direct amplification. It can also be where cleanup and/or amplification occurs. To minimize the duration of thermocycling and the amount of energy required, this second chamber can have minimal volume, perhaps ranging from 2 μl to 25 μl, although other configurations are practical.

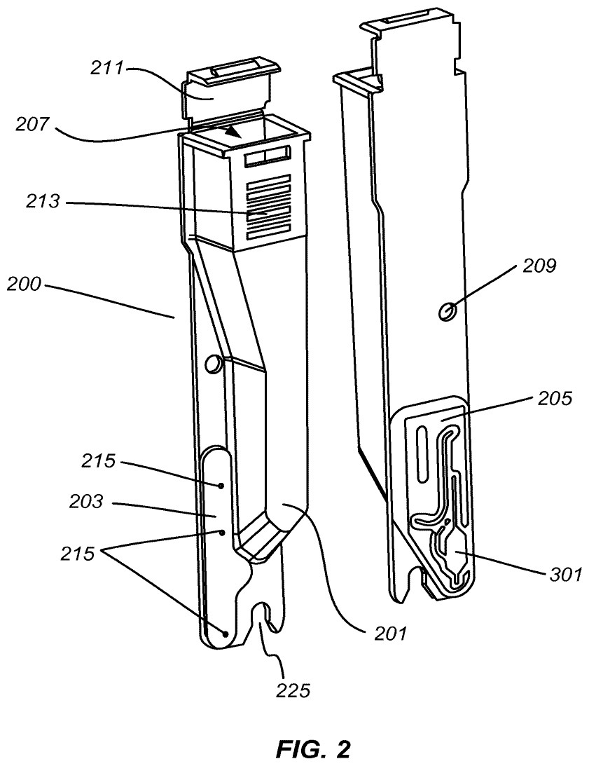

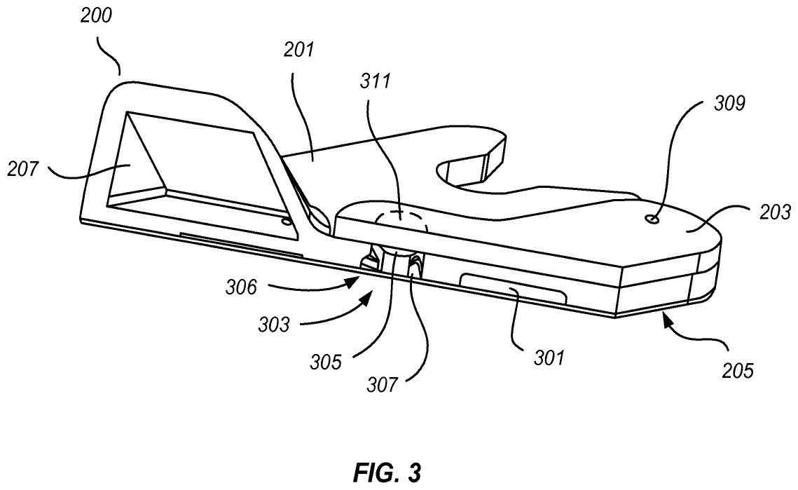

Referring to FIGS. 2 and 3, to an area of the cartridge body 201, a deformable film 203 can be bonded on one side, and a cover sheet 205, such as a plastic film or metal foil, can be bonded to the other.

The deformable material used in cartridges disclosed herein can be a plastic material (plastic deformation) or an elastic material (elastic deformation). The plastic material can comprise, without limitation, a polymer or a metal. Suitable plastic materials include, without limitation, polypropylene and polyethylene. Suitable metals include aluminum. Suitable elastic materials include, for example, elastomeric materials such as polysiloxanes, e.g., PDMS. Other deformable materials are further described herein.

In one embodiment, the deformable film serves as a gasket for three low-dead-volume connections. These provide an inlet, an outlet, and a purge line that can be used to flush out the cartridge and outlet line. The deformable film also serves as the flexible diaphragm for a valve. The valve seat 305 can be formed into the cartridge body. The inputs 306 and outputs 307 to the valve can be vias through the body, channels between the body and the deformable, or both.

The valve can be actuated by positive or negative pressure or applied to the deformable material over the valve seat. In another embodiment the valve can be actuated by a ram exerting mechanical force on cover sheet 205.

The deformable material may also fill a hole in the body, creating a flexible valve on the far side of the body. The deformable material can be pressed from the near side to deform through the body, and seal against a surface on the far side of the body.

In addition to being mechanically simple, structuring the cartridge around one molded body with functional elements on the surface increases the robustness. Leaks are critical problems, so the fluidic and pneumatic connections need to line up to enable sealing. Tolerance variations accumulate across assemblies, typically with each part contributing some variation. As a result, simpler assemblies can be more robust even with the same part tolerances. Furthermore, the single sample cartridge and other simplified cartridges in this instant disclosure involve only a few connections, further reducing the risk of leakage. Additionally, the effect of thermal expansion increases with size, so having few connections that are also close together reduces the risks still more.

This embodiment integrates parts, reducing materials and assembly costs. In one embodiment, it is designed with pneumatic ports, fluidic ports, and valve controls (either mechanical or pneumatic) onto one side. This simplifies connections and permits more space for other functions such as a temperature regulator (e.g., a thermocycler) to contact the cartridge, an optical system to interrogate the cartridge, or other measurement devices.

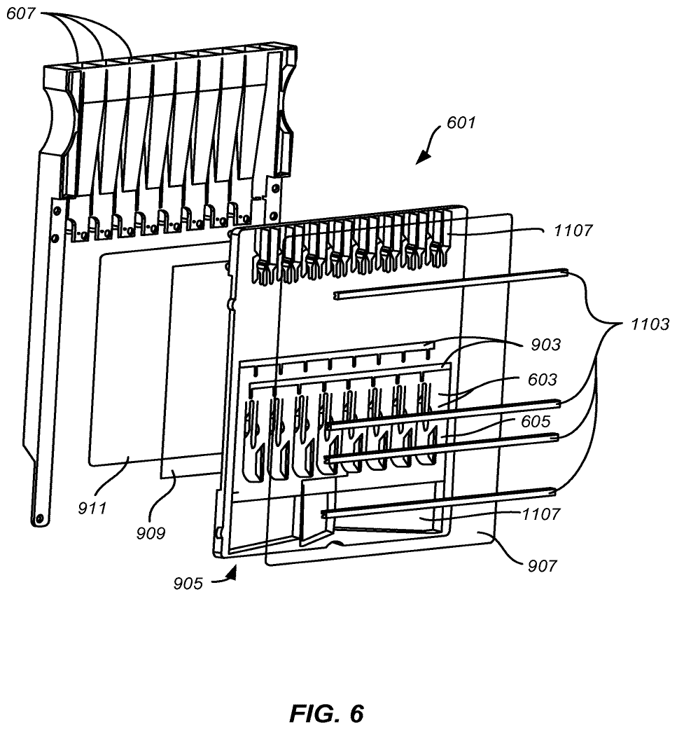

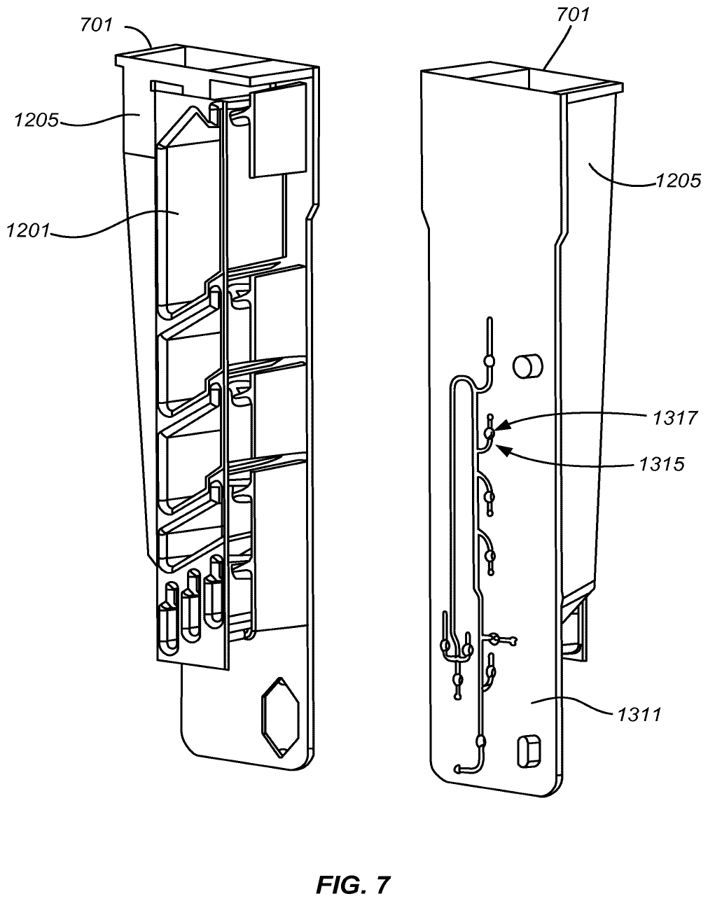

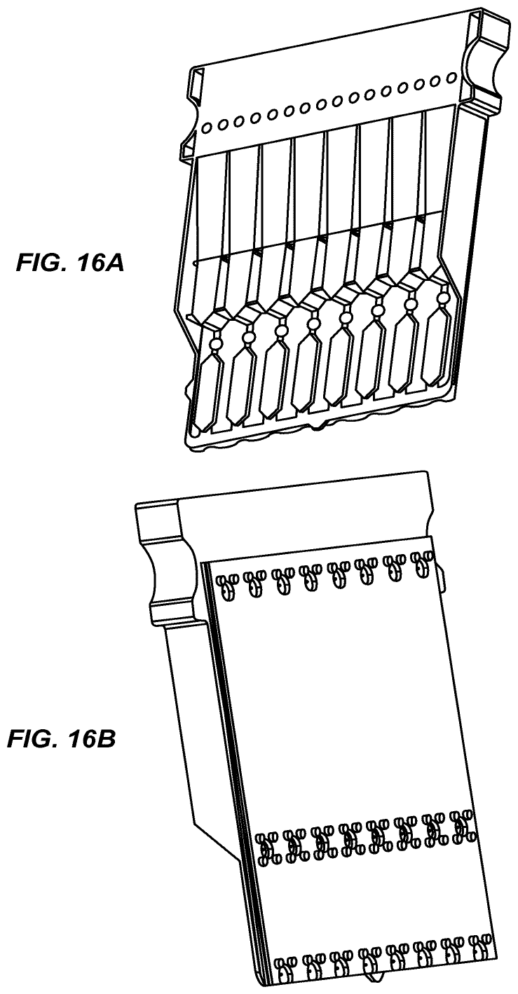

Cartridges constructed in this or other embodiments can also be built to accommodate multiple samples. These multi-sample cartridges can permit the operator to run multiple samples without having to insert multiple cartridges. (See, e.g., FIGS. 6 and 16)

Alternatively, single-sample cartridges can be assembled onto multi-cartridge holders. Operators running many samples at a time are able to take the rack containing used cartridges out of the system and insert the new rack containing unused cartridges. Operators running only a few samples are able to populate only as many spaces as they wish. The remaining spaces can be filled with dummy cartridges or left empty depending on the configuration.

The single-sample cartridges become particularly advantageous when paired with a latched cartridge interface 101 that can permit them to be inserted and removed singly. This can provide more flexible sample flow.

This interface can have a number of cartridge positions 103 or ‘slots’ that can open and close independently. They can apply a compressive force on the cartridges. They can be held closed by solenoids, or another means controlled by the software, or could be manually latched.

An operator with a new sample to run can insert the sample into a cartridge, and snap the cartridge top closed. He or she can then ask the system to open a slot. If a slot is not in use, the system opens it, permitting the sample to be inserted. If a processed cartridge is in the slot, the operator removes it. The operator could read the barcode, QR code, RFID or other identifying material on the cartridge before it is inserted or the system could read as it is inserted or afterwards. The operator can then push the interface closed, and enter additional information if necessary. The system can then start processing the sample immediately or start it automatically when next possible.

In an alternative embodiment, an operator with a new sample can manually open a slot or direct the system to open a slot. If a slot is not in use, the system opens it. If a processed cartridge is in the slot, the operator removes it. The operator inserts a cartridge. The operator could read the barcode, QR code, RFID or other identifying material on the cartridge before it is inserted or the system could read as it is inserted. The operator could then read the barcode, QR code, RFID or other identifying material on the sample if there is one and place the sample into the cartridge in the slot; alternatively the operator could enter sample tracking information manually into the system. After the sample has been added, the top of the cartridge can be closed by the operator or by the instrument. The operator can then push the interface closed manually or the instrument can close the interface. The system can then start processing the sample immediately or start it automatically when next possible.

This instant embodiment can be automated with a loading system that automatically inserts and removes cartridges as needed into slots. The automation can include mechanisms to load samples from a variety of sample types such as a collection of tubes containing swabs, microtiter plates containing liquid samples that can include preprocessing from single sources or mixtures, tubes containing liquid samples that can include preprocessing from single sources or mixtures, blood containers such as Vacutainers, or other containers for additional sample types.

The interface for each cartridge can float, permitting it to seal around the various fluidic and pneumatic connections on one side, while pressing the cartridge against the thermo regulator (e.g., thermocycler) 109 on the other. As shown, the floating interface rotates, pressing the cartridge against a fixed temperature regulator. Alternatively, multiple smaller thermocyclers could be used. These can rotate or translate, possibly pressing the cartridges against a common pneumatic and fluidic assembly.

After processing, the slot may remain closed to avoid contamination. The interface can press the cartridge against a temperature regulator, e.g., a Peltier device. This contact can be against the foil or film 205 enclosing the reaction chamber. Depending on the chemistry used, on the opposite side of the reaction chamber, the interface can house an LED, filter, and photodiode for reaction quantification or another detector.

In such an embodiment, when the reaction is a short tandem repeat (STR) reaction, in many jurisdictions for casework samples, the amount of human DNA must be quantified. The typical forensic process is to quantify an extracted sample using real time polymerase chain reaction (PCR) in a separate instrument before the sample is STR amplified. In this instant disclosure, a human specific probe is added to the STR mixture which has fluorescence outside the range used by the STR kit. The reaction chamber 301 is interrogated by a suitable wavelength of light for the human specific probe while the STR is being PCR amplified. The human specific probe can be a quencher such as a Black Hole Quencher® or a TaqMan® probe or other chemistries well known to one skilled in the art. As the PCR cycles increase, the fluorescence from the human specific probe is monitored to quantify the amount of human DNA in the reaction. In a preferred embodiment, the number of amplification cycles can be adjusted based upon the amount of human DNA measured; this can be on a cartridge-by-cartridge monitoring if independent thermal cyclers are in use. One advantage is that the human specific probe will allow the concurrent STR amplification to achieve an optimal amplification and produce an amount of STR product that is optimal for the kit regardless of amount of starting DNA in the sample. A second advantage is the real monitoring concurrent with the STR amplification allows integration of a sample-to-answer system without having an additional separate quantification process. A third advantage is for low copy number samples where there is barely enough sample to produce a good STR profile the integration of the quantification with the STR amplification prevents the aliquot typically used for quantification from causing the remaining sample to not have enough DNA for a successful STR amplification.

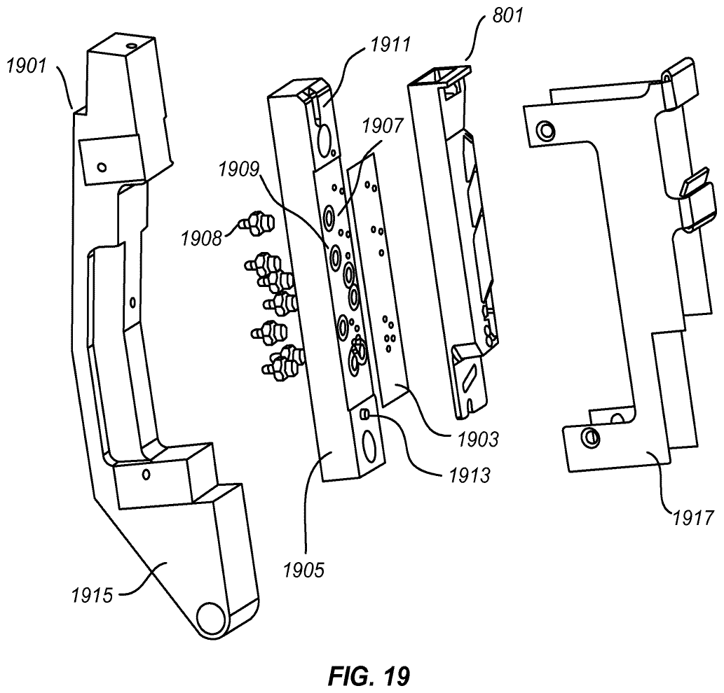

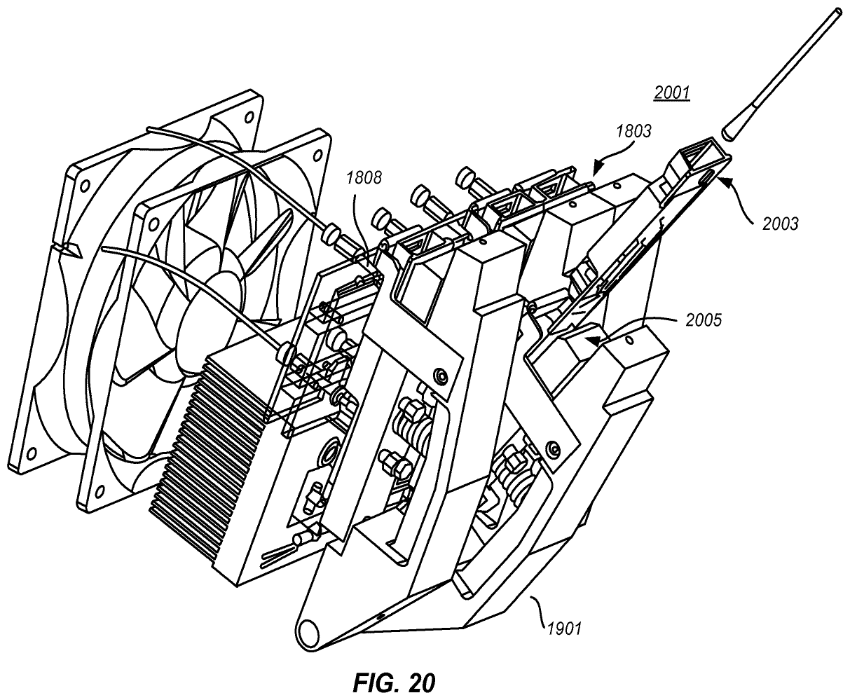

In addition to actuating the valve diaphragms (e.g., 311, 1301) mechanically, they can be actuated pneumatically. In one embodiment, the interface 1901 (FIG. 19) provides, for each valve, an interface diaphragm 1903 that conveys a pressure to the cartridge diaphragm 1301, pushing it against the valve seat 1317 to close the valve. The interface diaphragm is bonded to the interface block 1905 and encloses a threaded hole 1907 with a fitting 1908 to connect to the flexible tube carrying the pneumatic signal. Each hole can correspond to a valve, which it can close or permit to open, controlled by the pneumatic signal. The interface diaphragm may be silicone rubber bonded with RTV, with rings 1909 to limit delamination from fatigue. However, other deformable materials can be used.

The interface block 1905 is a component in the interface latch subassembly 1901. The block has alignment features 1911 and 1913 that mate to the cartridge alignment features 2003 and 2005 accurately locating the cartridge in the interface. The block mounts flexibly to a hinge arm 1915 that pivots to engage the cartridge to the interface, or permit the operator to insert or remove cartridges. A frame 1917 loosely guides the cartridge during insertion, ensuring that it can mate with the alignment features.

The single body cartridge allows on-chip storage/integration of reagent reservoir, including, for example, for example, capillary electrophoresis separation gel. This embodiment also permits STR manipulation without having reagents contact PDMS, which can interfere with certain biochemical reactions. This embodiment permits an integrated reaction chamber: The reaction chamber volume is defined by the outside of the fluidic layer and enclosed (e.g., by heat seal plastic, heat seal foil, graphite, etc.). It can connect to the circuit either by vias through the fluidics layer, or by enclosed channels along the surface.

In systems that use STR components that are sensitive to PDMS or other diaphragm materials, the second side can house the STR components in reservoirs 603 (FIG. 6) and use reaction chambers 605 separate from the PDMS layer 909. To improve room-temperature stability, the STR components can be stored separately. Vias through the fluidics layer may push or pull the STR components into the reaction chamber, without having the bulk of the STR mixture contact the PDMS or other membranes.

In addition to STR components, other reagents can also be stored on the second side of the fluidics layer. For laminated cartridges, which need to maintain a high degree of flatness near the pneumatic and fluidic circuits, these storage chambers could be above or below the laminate, or off to the side. The storage volumes would need vents near the top, and outlets near the bottom or narrowed sections capable of drawing the fluids upwards like a straw.

To minimize the risk of contamination from one sample to another, reagents that are used before amplification could have separate chambers above the laminated area. Reagents used after amplification, when contamination is less of a risk, can be shared among all sample circuits. This approach permits all reagents needed to run the system to be stored on a single cartridge.

Those reagents which require low pressures for movement or containment can be handled with diaphragm valves. Those reagents which require higher pressures, such as the separation gel, can be drawn out at low pressure into another chamber, and then pushed into the capillaries at high pressure.

### II. Cartridges Having a Fluid Distribution Channel

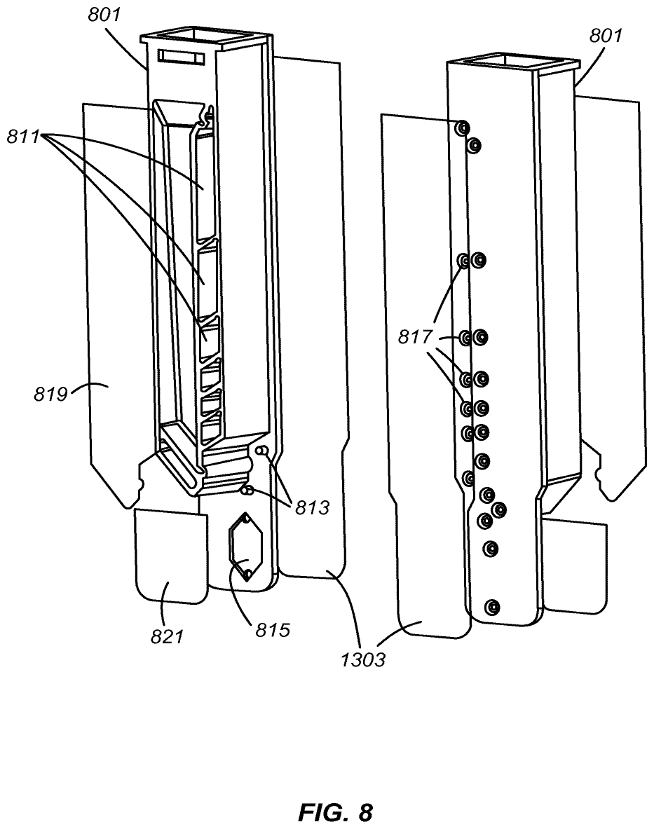

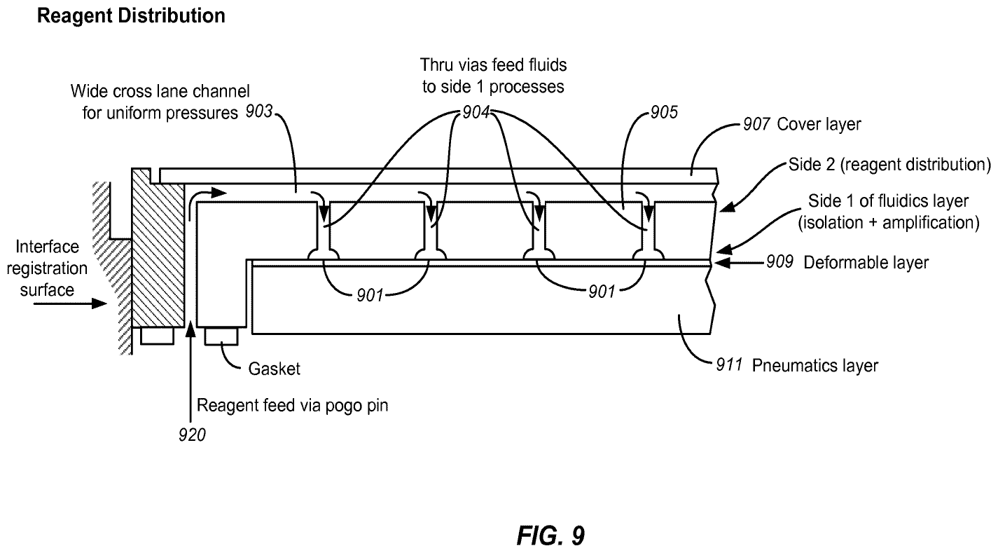

A double-sided fluidic layer offers a number of capabilities for multi-sample cartridges, such as an embodiment as shown in FIG. 6. For example, as shown in FIG. 9, if the circuits for individual samples are on one side of the fluidic layer, e.g., through channel 901, the other side of the fluidic layer could provide right-to-left channels, e.g., 903, to distribute reagents. Reagent distribution can otherwise require an additional fluidics part or external manifold.

### III. Pneumatic Channels to Selectively Block Diaphragm Valves

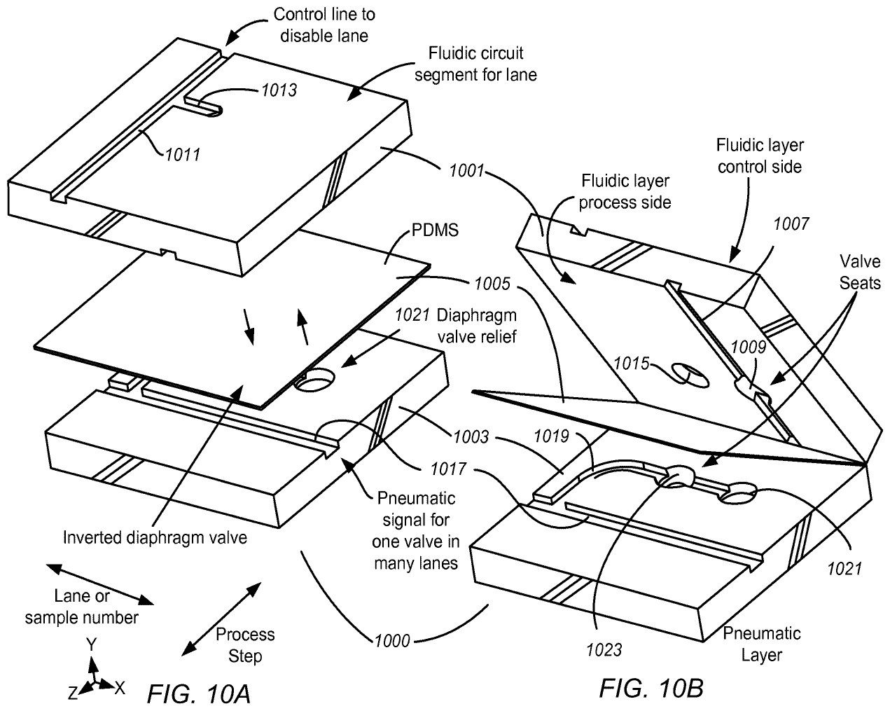

The right-to-left channels can also route pneumatic control signals to enable or disable specific circuits, as shown in FIG. 10A-B. This selective enabling or disabling of circuits can permit some samples to be run immediately, and other circuits to be reserved to run samples later.

### IV. Cartridges Having a Deformable Layer Sealed to a Plastic Body

Cartridges of this disclosure can have a body comprising a solid material. The solid material can be rigid, plastic (capable of irreversible deformation) or elastic (capable or reversible deformation). The body can be stiff or compliant. In some embodiments, the solid material is a polymer, e.g., a thermoplastic, such as polypropylene. The body can comprise an external surface comprising elements of fluidic circuits, such as channels, compartments, vias and valve seats. The body can be made by injection molding of the thermoplastic. These features can be covered with a layer of material attached to the surface of the cartridge body. The layer can function to seal otherwise open features such as channels and compartments. The material can be a deformable material that can deform to contact a valve seat, thereby closing the valve. In certain embodiments, the solid material is inelastic (not capable of elastic deformation). For example, the solid material is not an elastomer, such as PDMS.

The material can be attached to the surface of the body using a selective bonding process in which the material bonds to selected portions of the surface during the bonding process and does not bond to un-selected portions of the circuit after the bonding process is complete. For example, the material may bond to surfaces other than fluidic elements during the bonding process, and not bond to fluidic elements, such as channels and valve seats, after the bonding process. Methods for selective bonding include, for example, thermal bonding (e.g., heat sealing, welding, laser welding), chemical bonding (e.g., chemical bonding of oxide to PDMS) and selectively placed adhesives.

In one embodiment a layer of the deformable material is attached to a surface of a cartridge body through thermal bonding. This can include thermally bonding the material directly to the surface, or thermally bonding the material through an intermediate layer of material. In the latter case the material can be a laminate in which a deformable material is coated with a layer of material that contacts the surface and that melts at lower temperature. In either case bonding typically comprises contacting the deformable material to the body to form a combination and using a die to apply heat and pressure to the combination. Application of heat and pressure melts substrates in locations at which the material and body are in contact and fuse them, e.g., through coalescence. This process is more generally referred to as welding.

A material that bonds to a body through application of heat and pressure is referred to herein as “heat seal”. Heat seals are well known in the art and are commercially available. For example, 4titude (Walton, Surrey, UK) commercializes a variety of heat seals. These heat seals are described on the website 4ti.co.uk/sealing/heat-seals/. These include, for example, Clear Seal, Clear Weld Seal and Foil Seal. Heat seals also are produced by Axygen, a Corning brand (Corning, Tewksbury, MA, USA). These include Axygen® PlateMax heat sealing film and sealing film rolls. See the website: catalog2.corning.com/LifeSciences/en-US/Shoppi ng/Prod uct.aspx?categoryname=Genom ics+and+Proteomics(Lifesciences)% 7cPCR+Products(Lifesciences)% 7cSealing+Films+and +Tapes+for+Microplates(Lifesciences)% 7cHeat+Sealing+Films+and+Tapes+for+Microplate s(Lifesciences).

The deformable material can be a homogenous or non-homogenous material. In certain embodiments, the heat seal material is made from the same material as the body of the cartridge. It can comprise a thermoplastic (e.g., polypropylene, polyethylene, polystyrene, cycloolefin co-polymer (COC), mylar, polyacetate) or a metal (e.g., aluminum). See, e.g., WO 2012/136333. The heat seal can be produced by contacting a heat seal layer with the body and applying heat and pressure. Non-homogenous films include laminates having a first side for contact with the heater and a second side for contact with the body. The first side has higher melting temperature (“high melt”) than the second side (‘low melt ”). This permits use of a heat source to bring the lower side to its melting temperature before the first side allowing bonding to the body without bonding to the heater.

In the single sample cartridge, one side of the body into which compartments are formed is covered in a film or foil that can be adhered or thermally attached to the body. This encloses a second functional layer while only requiring one molded part. This permits functional details—valves, channels, etc. on different sides of the body. In the case of the single sample cartridge, this permits the valves, pneumatic connections, and fluidic connections to be on one side of the cartridge, while the reaction chamber is on a different side of the cartridge. As a result, the temperature regulator controlling the reaction chamber temperature can do so through a thin film, rather than the deformable gasket, which can result in quicker and more controlled thermocycling.

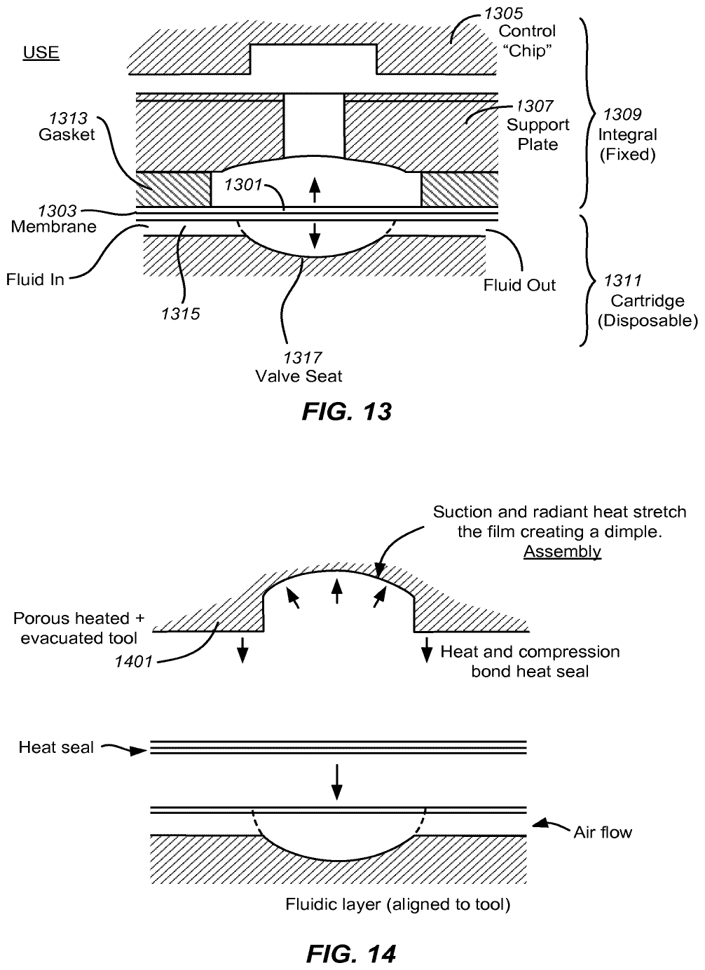

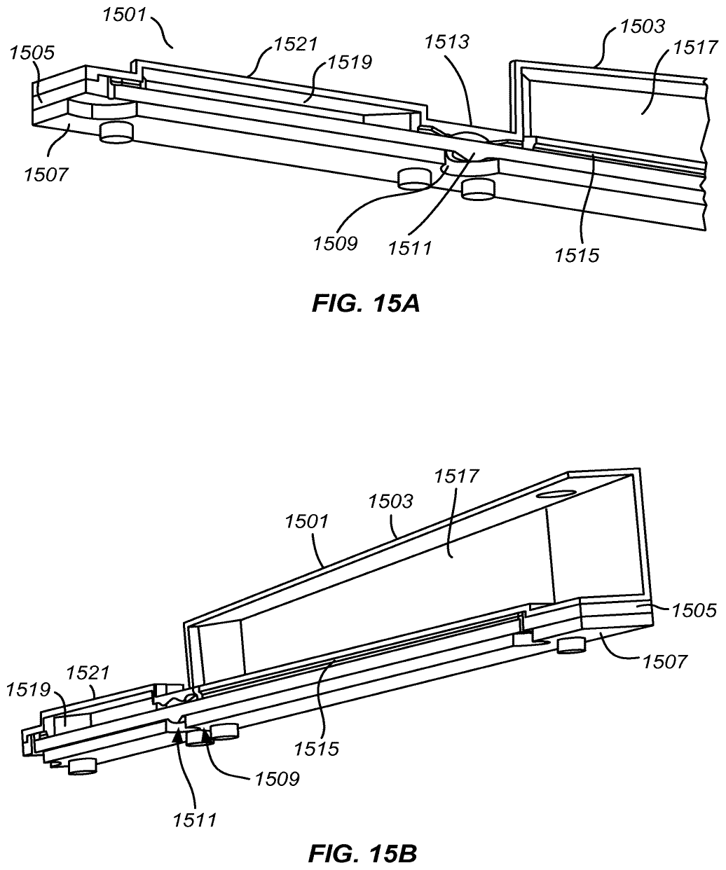

Referring to FIG. 13, in this embodiment, the valve diaphragms 1301 are formed by a film, such as a plastic film. These films are sealed to the cartridge body 1311, enclosing the fluidic circuit 1315. The sealing can be through a heat-seal, a pressure-seal, laser welding, chemical bonding, adhesive or other method well known to one skilled in the art. These valves can be actuated by a control circuit on the system 1305. However, the control circuit can be a permanent part of the system interface 1309 and need not be part of the disposable cartridge 1311. This control circuit can be mounted to a mechanical support plate, with through vias to conduct the pneumatic signals. Gaskets 1313 between the support plate 1307, control circuit 1305, and the cartridge will prevent leaks. In one embodiment the gaskets 1313 can be part of the interface 1309. In an alternative embodiment the gaskets 1313 can be part of the disposable cartridge 1311.

Depending on the film used, there can be a slight overhang around the perimeter of the valve, channel, or volume. This overhang can be due to adhesive or plastic flow during bonding. To prevent these from affecting the quality of the valve seal, the valve inlet, outlet, or both can be through vias in the valve seat 1317. The valve seat, away from the perimeter, can be less affected by the overhang.

Because of the limited flexibility of the film, it may be necessary to create a dimple over the valves. This can be achieved by coining the film downwards against the valve, with the limitation that the heat applied must not be enough to bond the film to the valve seat. A preferred approach would be to vacuform the dimples. The ordinary process of heat sealing involves applying a combination of heat and pressure to create a bond. If the heated tool (1401) was made from a porous material and had cavities cut above the valves, suction could be applied that can draw the film over the valve into the cavity, creating a dimple. This can occur at the same time as the film was being bonded to the body in other areas.

This embodiment can allow multiple fabrication and material options. For example, PDMS, which is commonly used in microfluidics, could be replaced with such a material, such as the heat seal films. This embodiment also reduces requirement for flatness in pieces, permitting other cartridge materials, such as polypropylene.

The use of the fluidics layer for reagent storage and the use of sections of the enclosing film for shipping as in the embodiment of 601, and the use of sections of the enclosing film to implement valves as in the embodiment of 701, permits the cartridge functions to be served by one molded piece and one or more bonded films. Another embodiment, as shown in FIG. 8, uses this construction.

### V. Clamp-Sealed Cartridges

FIG. 11 shows a section of the cartridge 601. By using raised lines or areas, e.g., ridges, 1101 to control the bonding of the film, vents can be built into the fluidic layer 905. These raised areas can provide a localized contact when bonding, controlling which areas are bonded and which areas are not, resulting in defined channels.

To close off the outlets 1109 and vents 1105 to these reagent chambers 1107, bar clamps 1103 can be built into the shipping container for the cartridge. These bar clamps can have some rigidity, but can be covered by a deformable or other material that can conform to the cartridge surface. It may have a basic shape or be formed to mate with the cartridge surface.

Bar clamp 1103 is able to hold the seal film cover (907, not shown in FIG. 11) against the body or fluidic layer 905, closing off the defined channels. After shipment but before use, the cartridge is removed from its packaging, which either removes the shipping clamps as the packaging is opened, or the clamps are removed separately from the cartridge after the cartridge is removed from the packaging.

If the flexible bar clamp is U-shaped as shown, it can close each channel in two places to prevent leakage. The operator will then be able to confirm that no leakage has taken place by examining the area between the two seals. Any leakage past both seals will generally leave a residual amount between the two seals.

Before use, the two vias leading to each of the reagent reservoirs are held closed by a shipping clamp. This shipping clamp can apply a uniform force to a flexible pad, causing the pad to deform and hold the valves closed. Alternatively, it can include a number of small rubber contacts that can individually hold each valve closed. This shipping clamp can then be removed before the cartridge is inserted into the system.

### VI. Diaphragm Valve With a Bossed Diaphragm

Diaphragm valves also can be actuated mechanically using a ram, e.g., a pin. These can be actuated by a solenoid. If actuated by solenoid, it may be beneficial to add a boss (such as element 1511) to the outside of the deformable. This permits a ram to push against the boss, creating a centered force sealing the valve, even if the solenoid is not centered over the valve.

### VII. Turret Cartridge

Cartridges actuated mainly by a syringe pump or by a manually operated syringe are included in this instant disclosure. The cartridges can be controlled by motors controlled by the computer on the system.

One embodiment of a cartridge utilizes a syringe pump for actuation, with selectable, specialized areas arranged in a ring. These areas can each store reagents, house the swab or punch, contact a temperature regulator, connect to the capillary for separation, etc.

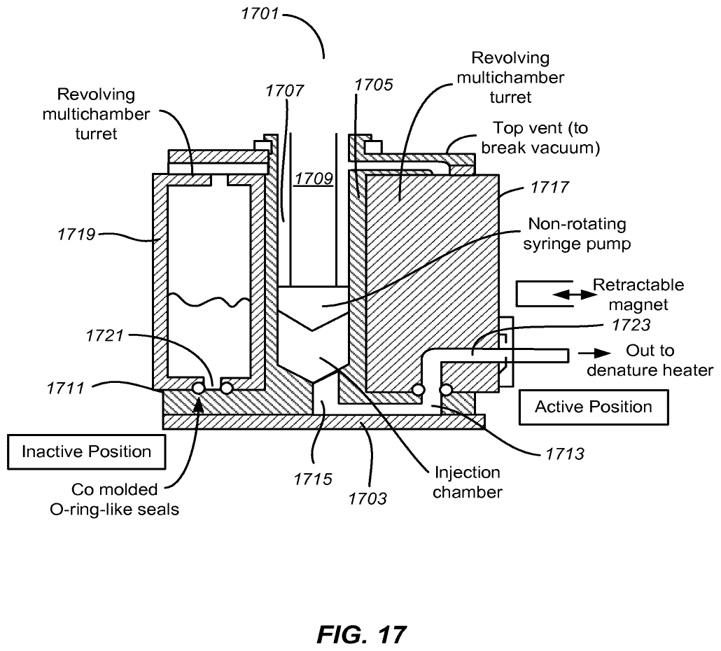

Referring to FIG. 17, the cartridge can implement a rotary selector valve, either by rotating the cartridge body 1717 or an internal valve. By rotating, various inputs or outputs can be selected. This rotation can be driven by, for example, a stepper motor. The syringe 1709 can in turn be driven by for example, a linear stepper motor. This permits a broad range of general functions to be controlled by two stepper motors. The interface can also make use of one or more temperature regulators. Thermocycling can be implemented by cycling the temperature of a temperature regulator, or by rotating to contact one of multiple controlled heat sources to reduce the power usage and may increase thermocycling speed. It can also have an LED, filter, and photodiode for reaction quantification.

One, two or three positions on the hub can be temperature controlled. One position on the hub can be open on top, for sample insertion. One or more positions can have external, retractable magnets.

Turret cambers can include: (A) Vent: air to injection chamber; (B) Vent: to denature heater; (C) lysis chamber/swab vial; (D) lysis buffer/Waste; (E) mix chamber/beads; (F) water; (G) ethanol; (H) STR lyosphere (amplification reagents); (I) capture solution and size standard (or lyosphere); (J) eluting agent; (K) electrophoresis separation gel; (L) reaction chamber.

Gel injection may be to a booster pump instead of directly to the capillary. This would avoid the need for high-pressure seals, in the sample cartridge (This would permit gel injection in parallel with other functions.) If the capillary can be mounted directly, denature heading may be complete by one of the heated positions, without an external denature heater. An external waste gate, at the cathode end of the capillary may still be necessary.

This embodiment can permit an interface consisting of one rotary and one stepper motor, eliminating pneumatic pumps, manifolds, anode module/gel filling mechanism, etc.

### VIII. Lead-in Guiding Fluid Delivery Pogo Pin

FIGS. 18A and 18B show a low-dead-volume floating connector 1808 in cross-section. When the interface is open as shown in FIG. 18A, the pogo is forced down against the home lead-in 1801 by a spring 1802. This will reset the pin to a consistent home position relative to the pogo block 1803. When the interface closes onto a cartridge that is off-center, the pogo contacts the engagement lead-in 1804 and is pushed up, freeing the engagement play 1805. The engagement lead-in then guides the cartridge within this play. Once aligned, the conic surfaces of the pogo pin 1806 and cartridge 1807 connect. The slight taper magnifies the force of the spring, creating a seal. This seal requires some flexibility in the cartridge. Since the surrounding wall thickness is driven by the engagement lead-in, this limits the lead-in size. The engagement lead-in 1804 and the engagement play 1805 will both need to be large enough to accommodate all manufacturing and other tolerance variations. If the pogo pins did not self-reset to a consistent home position, the engagement lead-in can changed to accommodate variations from the engagement play as well.

### IX. Vent Tabs

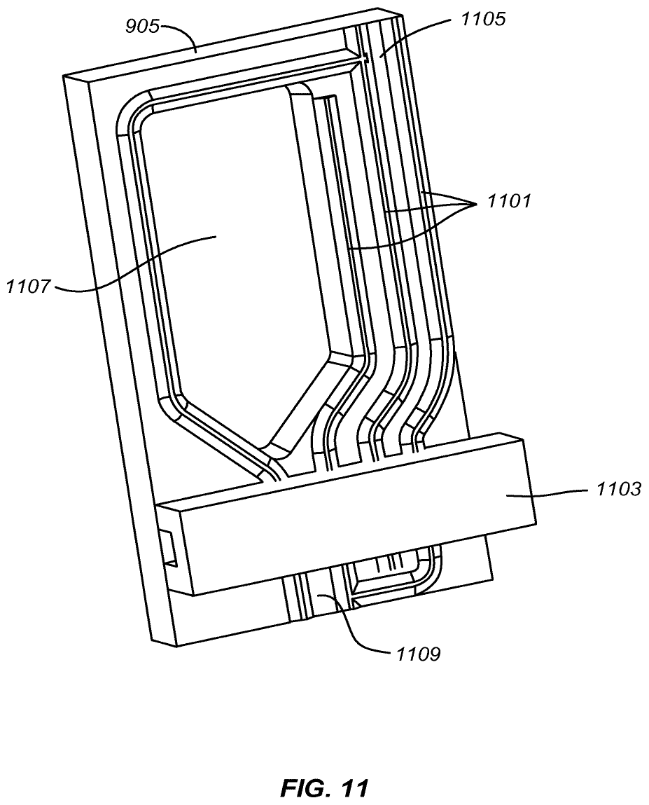

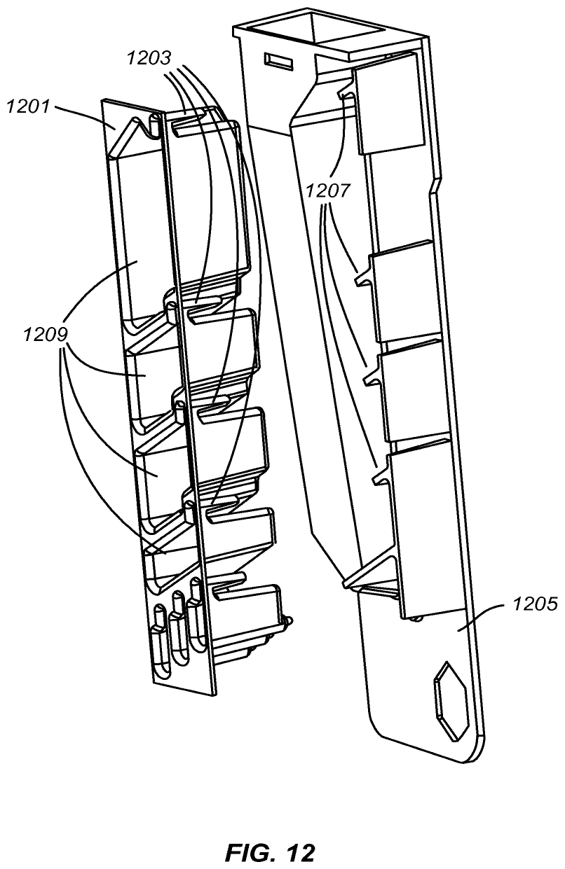

In an embodiment shown in FIG. 12, the cartridge includes two injection molded plastic parts, a cartridge body and a reagent reservoir. When in use, the reagent reservoir can be pressed against the body. This can snap open the vents, and engage connections between the body and the reservoir.

For cartridge concepts that have bodies 1205 and moveable reagent reservoirs 1201, such as the cartridge of the embodiment of 701, there is another approach to providing vents. This approach does not require additional parts. This is to build in designed-to-fail tabs (e.g., 1203) into the reagent reservoir. Before use, these tabs will remain closed, but will have a slight interference with the cartridge body 1205. When the reagent reservoir is engaged by pressing, these tabs will try to pull away from the main volume. It will tear or crack, opening a small vent in the reservoir.

This embodiment can provide a vent for on-cartridge reagent reservoirs without requiring additional degree of freedom in the interface or additional part in the cartridge.

### X. Fluidic Device With Diaphragm Valve

The cartridge can utilize off-cartridge pumps to move liquids. To avoid the need for high mechanical precision, these valves and channels can be larger than traditional microfluidic valves and channels.

The cartridge of this disclosure can include diaphragm valves. Diaphragm valve can be formed having a valve chamber in the fluidics layer of the cartridge and a deformable membrane attached to a surface of the fluidics layer and providing a diaphragm for opening and closing valve. In one embodiment, the valves are normally open. That is, at ambient pressure the valve is open and closing the valve involves applying positive pressure to the diaphragm opposite the valve seat. Applying negative pressure to the diaphragm opposite the valve seat can further open the valve. The diaphragm can be actuated by pneumatic or mechanical pressure. In an embodiment of this disclosure the diaphragm is mechanically actuated by positive pressure applied by a ram or rod having an end configured for insertion into the valve chamber. In certain embodiments the rod has a compliant end that promotes contact between the diaphragm and a valve seat, thereby sealing the valve closed. Withdrawal of the rod relieves pressure on the diaphragm, thereby opening the valve.

In one embodiment of a normally open valve, a surface of the fluidics layer comprises a recess that both defines a valve chamber and functions as a valve seat. At ambient pressure the deformable membrane does not sit against the valve seat and the valve is in an open configuration. Positive pressure on the deformable membrane from the side opposite the fluidics layer pushes the deformable membrane against the valve seat, closing the valve. The valve seat can take a curved shape that is convex with respect to the surface of the fluidic layer, against which the deformable membrane can conform. For example, the valve shape can be a section of a sphere or an inverted dimple or a dome. Such a configuration decreases the dead volume of the valve, e.g., by not including a valve chamber that contains liquid while the valve is closed. This valve also comprises a surface against which the deformable membrane can conform easily to close the valve. In another embodiment, the concave surface can comprise within it a sub-section having a convex surface, e.g., an inverted dimple comprising an extraverted dimple within it forming, for example, a saddle shape. The convex area rises up to meet the deformable membrane under pressure, creating a better seal for the valve.

Valve seats can be recessed with respect to the rest of the surface by about 25 microns to about 1000 microns, e.g., about 700 microns. Valves can communicate with fluidic channels that are either microfluidic or macrofluidic (e.g., having an aspect less than 500 microns or having an aspect greater than 500 microns or at least 1000 microns). In certain embodiments of a normally open valve, the concavity is recessed less than the channels to which it is connected. In certain embodiments the channels can enter partially into the concavity, for example forming a vault. In certain embodiments, the channels and concavity are formed by micromachining, injection molding or embossing.

### XI. Valve Actuated by Ram With Compliant End

One embodiment involves closing a fluidic valve with a ram. The valve can be comprised in substrate that contains the valve and one or more input and output fluidic channels. There can be more than one input and output. These channels can enter the surface of the dome valve at any location on the surface as long as there is a sealing surface between channels. In certain embodiments, channels can enter the valve chamber through vias that connect with the channels. The dome valve is then covered with a membrane either elastic or non-elastic film. The film is affixed to the perimeter of the dome to create an air and liquid tight seal. The ram is then pressed against the film diaphragm with sufficient force to deform the diaphragm and press the film onto the dome surface. The pressure from the ram creates a fluidic seal between the orifices of the ports entering the dome valve.

In one embodiment the valve is configured as a router. The router can have, for example, four inlets/outlets. In this configuration the forked ram, when engaged, can block access to the router by some, but not all, of the inlets/outlets. For example, the forked ram could allow fluid flow through the router in a north-south direction or not it in an East-West direction.

The ram is structured such that there are one or more flexure posts defining an identical dome surface to match the valve dome surface with the offset of the thickness of the diaphragm. The flexure posts with the seal seat tips will be able to self-align to the target seal areas of the dome, namely the perimeter of the orifice for the input and output channels of the valve. The flexure posts also concentrate the stress generated by the force applied to the overall post onto the active seal area.

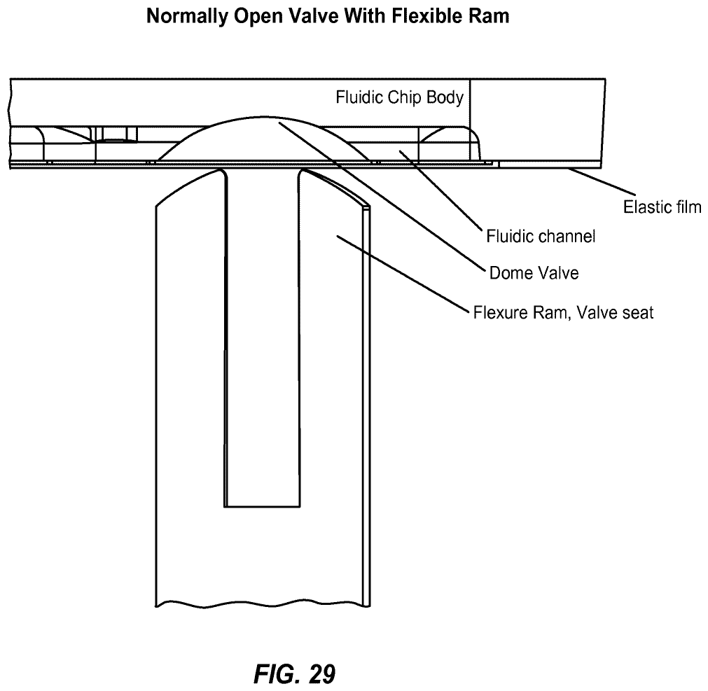

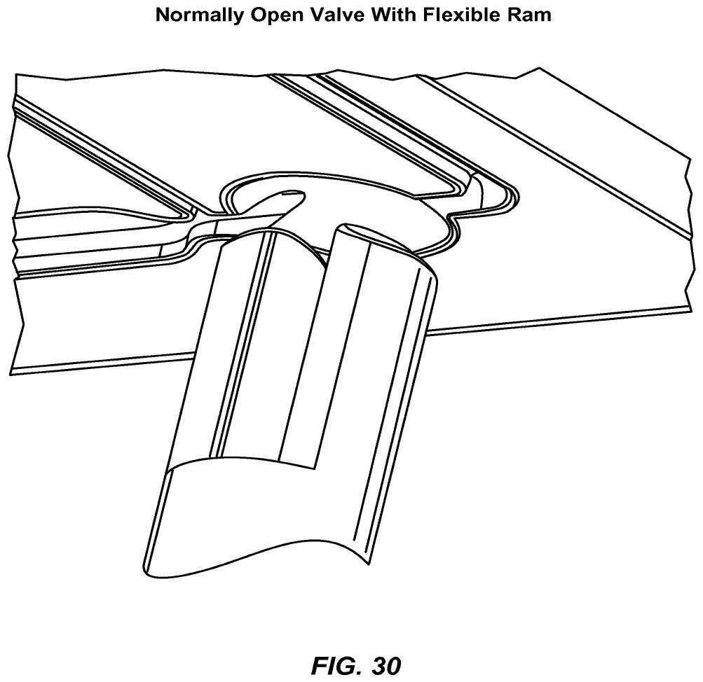

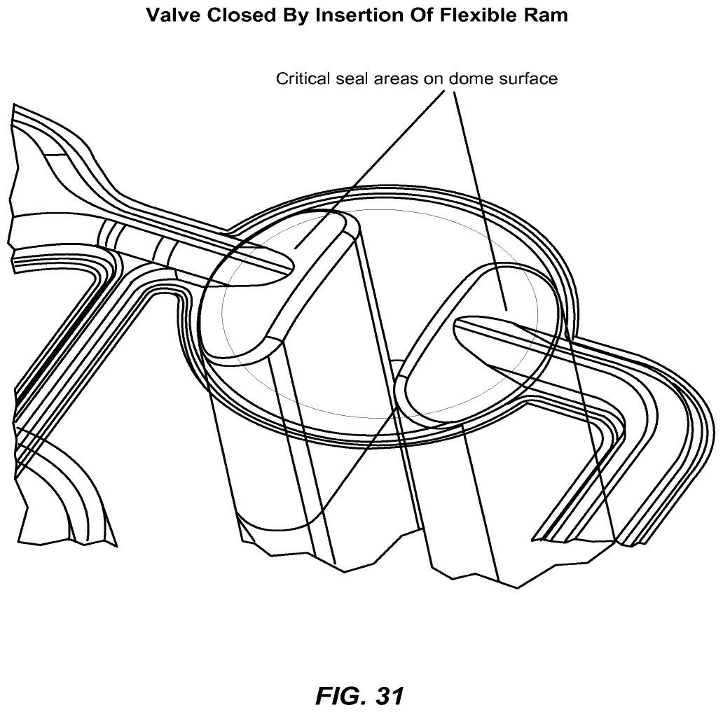

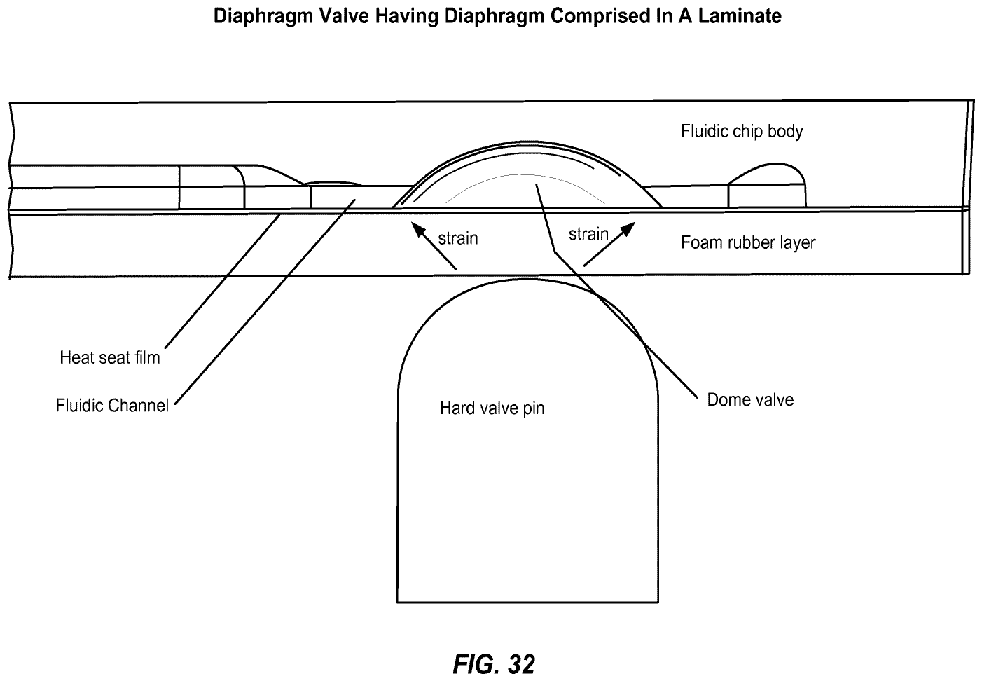

Referring to FIGS. 29 and 30, a fluid chip body comprises a recess forming a valve seat (“dome valve”). The recess defines a space that functions as a valve chamber. The fluidic chip body also includes fluidic channels (which can be microfluidic channels) in fluid communication with the valve through inlets and outlets. A surface of a fluidic chip body into which the recesses impose is overlaid with a deformable membrane (“elastic film”). A ram actuates the valve by applying pressure to deformable membrane. The ram can include a fork or slotted end that provides compliance to the flexure posts tines of the fork. An end of the ram has a form that complies with the shape of the valve seat. Referring to FIG. 31, when pressed against the deformable membrane, the ram deforms the deformable membrane against the valve seat. By contacting the valve seat around the valve inlet and valve outlet, the diaphragm closes the valve, preventing fluid flow through the valve. Relieving pressure on the diaphragm by withdrawing the ram allows the deformable membrane to assume its neutral position, opening the valve to fluid flow. The ram can be actuated, for example, by a solenoid.

### XII. Reaction Chamber

In one embodiment a fluidic device of this disclosure comprises a reaction chamber that comprises a solid substrate, e.g., solid phase extraction material, for retaining analyte from the sample. The solid substrate can comprise a material that binds the analyte, such as a nucleic acid such as DNA. The amount of solid substrate in a chamber and the selected to retain the predefined amount of analyte. For example, the material can be a Whatman FTA paper or a carboxylated material. Alternatively, the solid substrate can be an absorbent or sponge-like material that absorbs a predetermined volume of fluid. The material can be in the form of a monolith. The material can be, for example, PVDF (polyvinyldiene fluoride) membranes, filter paper, glass particles, silica, or other solid phase extraction material. In operation, lysate is pumped through the chamber and a predetermined amount of analyte is retained on a solid substrate. Then, retained material is contacted with reagents, e.g., reagents for PCR. The resulting material can be incubated to form a reaction product. For example, the chamber can be put into thermal contact with a thermal-control device, such as a Peltier, and the reaction mixture can be thermal cycled. In another embodiment, the chamber can include a pocket or container designed to retain the defined volume of liquid.

### XIII. Contaminant Deactivation

In one embodiment the fluidic layer includes a waste chamber. A waste chamber can contain material that degrades nucleic acids, polypeptides, or other analytes. For example a material can comprise a chlorinated material, such as calcium hypochlorite. Alternatively, the waste chamber can include in absorbent material that absorbs waste containing liquid

In another embodiment the nucleic acid degrading material is contained in a water-soluble capsules in yet another embodiment the nucleic acid degrading material is combined with an absorbent material such as cellulose or polypropylene fibers.

In another embodiment, the waste chamber contains enzymes that degrade the nucleic acids (e.g., nucleases), polypeptides (e.g., proteases), or other analytes such as phosphorylated sites (e.g., phosphatases).

### XIV. Cartridge and Method

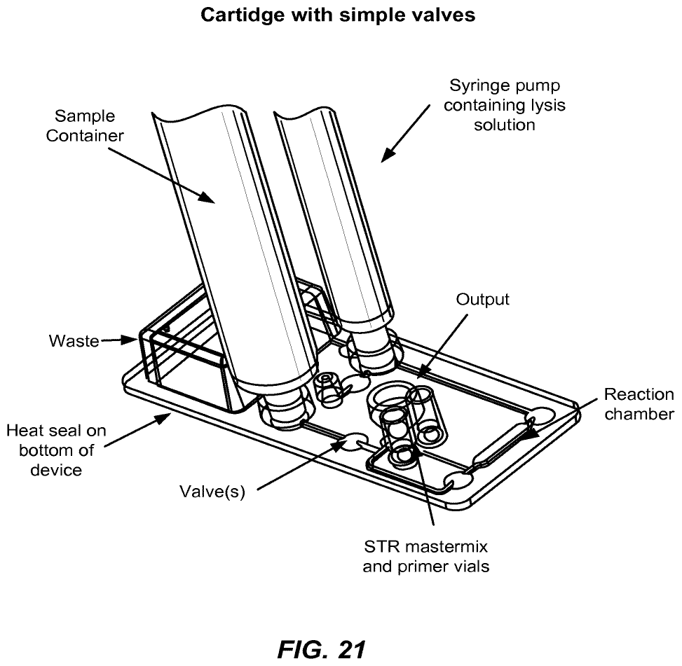

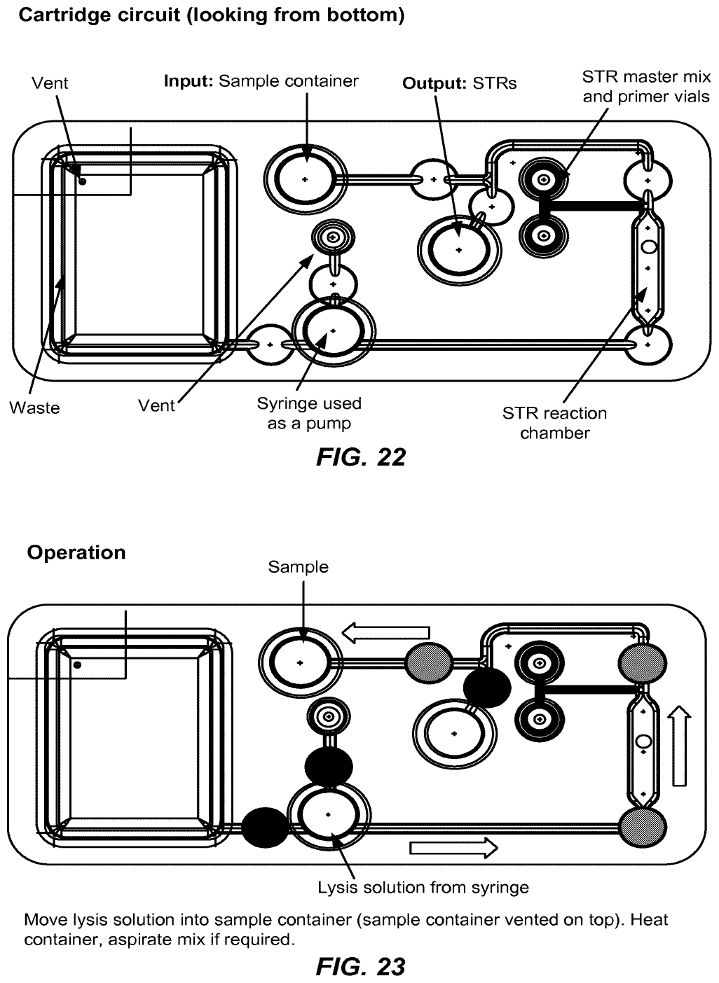

FIGS. 21 and 22 shows a fluidic cartridge configured for extracting nucleic acid from a sample, performing amplification on the sample, and outputting the amplification product. The cartridge includes a port configured to accept a sample container adapted to receive a sample, such as a swab; a port configured to accept a syringe pump containing or connected to reagents, such a lysis solution; a port configured to accept receptacles separately carrying PCR master mix and PCR primers; a reaction chamber, e.g., for thermal cycling; a waste chamber; a vent; an output port; fluidic channels (which can be microfluidic or microfluidic channels) in communication with these elements; valves for regulating flow of fluids in the fluidics circuit all of the cartridge. The valves can be, for example, diaphragm valves.

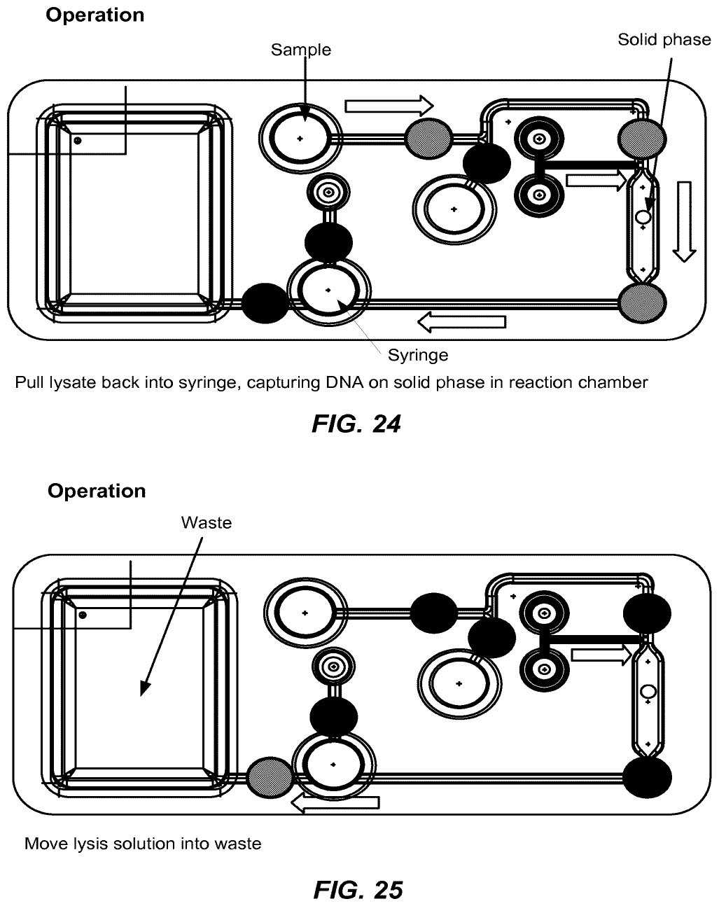

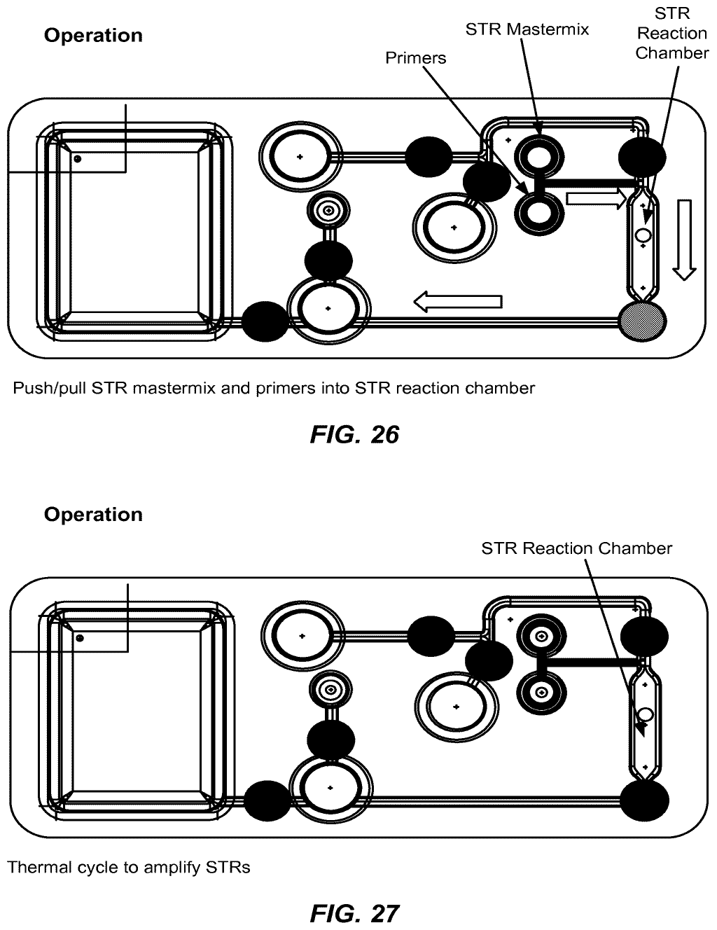

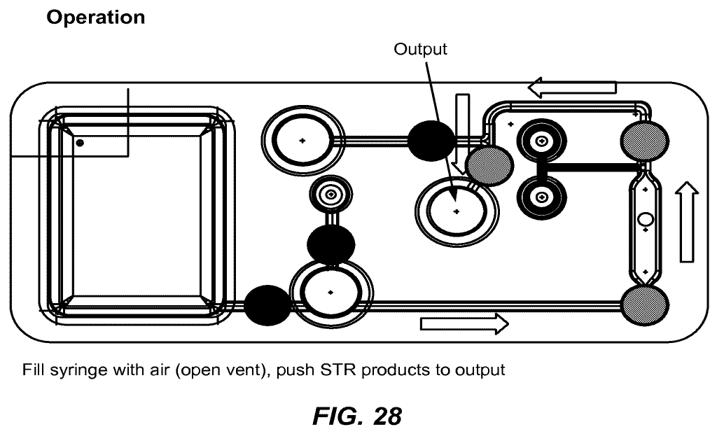

FIG. 23 shows the operation of a cartridge. Closed valves are indicated in darker shade, open valves are indicated in lighter shade. Arrows indicate the flow of liquids which are moved by the syringe. Lysis solution from the syringe is moved through a fluidic channel into the container containing a sample. The sample can be heated or sonicated to facilitate cell lysis. In FIG. 24 lysate is pulled back into the syringe and nucleic acid is captured on a solid phase in the reaction chamber. In FIG. 25 lysis solution is transported into the waste chamber. In FIG. 26 PCR master mix and primers, which can be contained in separate containers, are moved into the reaction chamber, for example pushing the liquid from one side as the syringe provides vacuum from another side. In FIG. 27, the reaction chamber is subjected to thermal cycling to amplify target sequences, for example, STR sequences, while all the valves are closed. In FIG. 28 the amplification product is moved to an output port where it can be transferred for further analysis.

### XV. Integrated System

The cartridges of this disclosure are useful in integrated and automated sample-to-answer systems that, starting from a sample comprising biological material, generate an analysis of the sample. In certain embodiments, the biological material is DNA and the genetic profile involves determining one or a plurality of alleles at one or a plurality of loci (e.g., genetic loci) of a subject, for example, a STR (short tandem repeat) profile, for example as used in the CODIS system. The system can perform several operations, including (a) extraction and isolation of nucleic acid; (b) amplification of nucleotide sequences at selected loci (e.g., genetic loci); and (c) detection and analysis of amplification product. These operations can be carried out in a system that comprises several integrated modules, including an analyte preparation module; a detection and analysis module and a control module.

Systems provided herein may be fully integrated. Sample processing can be accomplished in a single system without having to remove a sample and transfer it to another system. Systems provided herein can be fully automated, enabling a user to process a sample without substantial input from the user.

A sample preparation module includes a cartridge module assembly configured to engage and operate one or more than one sample cartridge. A sample cartridge is configured to receive one or more samples and to perform nucleic acid extraction and isolation, and DNA amplification when the cartridge is engaged with a cartridge module assembly in the system. It can also include controls and standards for assisting in analysis. In other embodiments, a sample cartridge is configured to receive one or more samples and to perform cell lysis, and enzymatic assays when the cartridge is engaged with a cartridge module assembly in the system.

The sample preparation module can include a receptacle for receiving one or more cartridges, an engagement assembly to engage the cartridge; a fluidic manifold configured to engage ports in a cartridge and to deliver pressure and/or fluids to the cartridge through the ports; a delivery assembly configured to deliver reagents, such as amplification pre-mix, from a compartment in the sample cartridge to an amplification compartment; a pneumatic manifold configured to engage ports in a cartridge and to deliver positive or negative pressure to the cartridge through the ports for moving fluids and operating valves, pumps and routers in the cartridge; a pump configured to deliver pressure to the fluidic and pneumatic manifold. Consumable reagents can be carried in a module, e.g., a buffer module, that is, removably engageable with the cartridge module.

PCR can be carried out using a thermal cycler assembly. This assembly can include thermal controller, such as a Peltier device, infrared radiation source, resistive heating element, circulating water or other fluids, circulating air, movement of constant temperature blocks, or other material, which can be configured to heat and cool for thermal cycling and can be comprised in the cartridge module which can be configured to move the thermal controller into thermal contact with the thermal cycling chambers, for example, through a heat spreader (or thermoconductor that can spread/distribute heat and cooling) disposed over each of the reaction chambers. In some embodiments, the cartridge comprises a temperature regulator assembly having one or more (e.g., 2, 3, 4, 5, 6, 7, 8, 9, 10, 16, 24, 32, 40, 48 or more) thermocycling chambers and the sample cartridge can be in fluid communication with a fluidic channel.

An analysis and detection module is configured to receive analyte from the sample preparation module and perform capillary electrophoresis on the analyte to detect analytes separated by electrophoresis and to analyze the detected analytes. It can include a capillary electrophoresis assembly, a detection assembly, and an analysis assembly.

The capillary electrophoresis assembly can include an injection assembly, that can include a denature heater assembly, a positioning assembly for positioning an analyte for capillary injection; a cathode assembly; a capillary assembly; an anode assembly; a capillary filling assembly for filling a capillary with separation medium and a power source for applying a voltage between the anode and the cathode.

A detection assembly can comprise a laser configured to illuminate the capillaries and a detector. The laser can be configured to excite fluorescent dyes in the analyte. In alternative embodiments, the laser can be replaced by an alternate light source such as an LED. The detector can include a CCD array, photomultiplier, diode array, or other detector, for detecting light produced by excited dyes and for producing an output signal.

An analysis assembly can include a computer comprising memory and a processor for executing code (e.g., code on a tangible medium) for analyzing the output signal and producing a computer file containing an analysis of the signal. Such an analysis can include, for example, identification of alleles from various STR loci. The computer file can be in a format that is compatible with public databases. For example, the file can be in CODIS format which is compatible with the National DNA Index System (NDIS) operated by the FBI.

The system can be operated by a control module. The control module can include a user interface configured to receive instructions from and deliver information to a user. It can include software programmed to execute routines for performing the operations mentioned, above, and transmit and receive information, such as computer files, from remote locations, for example, over the internet.

### XVI. Method of Use

The cartridges of this disclosure can be used in an integrated system for preparing a sample, for example, DNA isolation and amplification. For example, in one embodiment, a sample contained on for example a swab or a card punch, can be introduced into sample chamber 207. The chamber can be snapped shut by the lid 211. The cartridge can be engaged with cartridge interface 103. Cell lysis buffer contained in an on-system reservoir can be feed through line 405 through interface assembly 401 into the fluidic channel in the cartridge and into the sample chamber 207. After lysis, lysate can be moved through a fluidic channel on the chip, for example, which pumps the fluid into a reaction chamber 301. In one embodiment, the DNA reaction chamber can include magnetically attractable particles that bind DNA and that can be immobilized in the reaction chamber by applying a magnetic force generated in the interface. This can eliminate the need for an intermediate DNA isolation chamber. Waste fluid can be moved through the cartridge and out through a vent. Reagents for performing PCR or other reactions can introduced into the reaction chamber through one of the fluid lines 405 connected to the interface. A thermal control mechanism in the system can apply heat to perform thermal cycling in a thermal cycling chamber 301 of the cartridge. In some embodiments the heat is applied to a heat transmission element, for example, a foil or metalized film, that improves thermal contact and transmission.

The cartridges of this disclosure can be used in an integrated system for analyzing a sample, for example, DNA isolation and amplification with real time or end point detection. For real time measurement, the samples can be interrogated by an optical detection system while amplifying in reaction chamber 301. The readout can be the change in fluorescence or by melting point. The probes can be human specific for human identification, forensics, or molecular diagnostic applications, or specific for pathogens for molecular diagnostic applications, or for bioagents for biodefense applications or nonspecific intercalators for determining amount of DNA present. Amplification methods include, for example, thermal or isothermal amplification reactions, for example, PCR, rolling circle amplification, whole genome amplification, nucleic acid sequence-based amplification, and single strand displacement amplification, single primer isothermal linear amplification (SPIA), loop-mediated isothermal amplification, ligation-mediated rolling circle amplification and the like

The cartridges of this disclosure can be used in an integrated system for analyzing a sample. The assay can detect a polypeptide (e.g., immunoassay) or a nucleic acid (e.g., PCR). The assay can be multiplex or single analyte. They can involve any assay to measure presence, amount, activity, or other characteristics of the sample. These include assays that involve detection by fluorescence, luminescence, chemiluminescence, Raman, absorbance, reflectance, transmittance, birefringence, refractive index, colorimetric and combinations thereof. In this instant disclosure, the enzyme master mix and the substrate might be individually added to the reaction and the progress or endpoint of the assay monitored optically.

For STR applications, after thermal cycling, other reagents such as molecular weight markers (size standards) can be combined with the PCR product. Movement through the cartridge can be controlled when diaphragm valve 303 is actuated by pneumatic or mechanical actuators wherein forces transmitted through line 407. Products of the PCR can be moved off chip for analysis through an output line.

While preferred claims of the present invention have been shown and described herein, it will be obvious to those skilled in the art that such claims are provided by way of example only. Numerous variations, changes, and substitutions will now occur to those skilled in the art without departing from the invention. It should be understood that various alternatives to the claims of the invention described herein may be employed in practicing the invention. It is intended that the following claims define the scope of the invention and that methods and structures within the scope of these claims and their equivalents be covered thereby.

1.-9. (canceled)

10. A cartridge comprising:
a substrate comprising a fluidic layer, a pneumatic layer, and a deformable layer between the fluidic layer and the pneumatic layer,
wherein the fluidic layer comprises:
a first side contacting the deformable layer and comprising a valve feature,
a second side opposite the first side,
a first fluidic channel,
a via in fluidic communication with the first fluidic channel and the valve feature, and
a second fluidic channel,
wherein the pneumatic layer is configured to transmit positive or negative pressure so as to control engagement between the deformable layer and the valve feature and thereby control fluidic communication between the via and the second fluidic channel in the fluidic layer.

11. The cartridge of claim 10, wherein the substrate further comprises a cover layer covering the first fluidic channel.

12. The cartridge of claim 10, wherein the pneumatic layer is configured to transmit the positive or negative pressure to selectively deform the deformable layer and thereby control engagement between the deformable layer and the valve feature.

13. The cartridge of claim 12, wherein the pneumatic layer is configured to transmit positive pressure to selectively engage the deformable layer with the valve feature and thereby prevent fluidic communication between the via and the second fluidic channel in the fluidic layer.

14. The cartridge of claim 12, wherein the pneumatic layer is configured to transmit negative pressure to selectively disengage the deformable layer from contact with the valve feature and thereby provide fluidic communication between the via and the second fluidic channel in the fluidic layer.

15. The cartridge of claim 10, wherein the pneumatic layer comprises at least one pneumatic channel configured to transmit pressurization fluid.

16. The cartridge of claim 10, wherein the valve feature is a valve seat.

17. The cartridge of claim 10, wherein the deformable layer and the valve feature form a diaphragm valve.

18. The cartridge of claim 10, wherein the first fluidic channel is located on the first side of the fluidic layer.

19. The cartridge of claim 18, wherein the second fluidic channel is located on the second side of the fluidic layer.

20. The cartridge of claim 10, wherein the second fluidic channel is located on the second side of the fluidic layer.

21. The cartridge of claim 10,
wherein the first fluidic channel and the second fluidic channel extend along a first dimension of the fluidic layer, and
wherein the via extends transverse to the first fluidic channel and to the second fluidic channel.

22. The cartridge of claim 10, wherein the valve feature is a first valve feature and the via is a first via, and wherein the fluidic layer further comprises:
a third fluidic channel,
a fourth fluidic channel,
a second valve feature, and
a second via in fluidic communication with the third fluidic channel and the second valve feature,
wherein the second valve feature is configured to selectively place the second via in fluidic communication with the fourth channel.

23. The cartridge of claim 10, wherein the fluidic layer further comprises a chamber in fluidic communication with the second fluidic channel.

24. A system comprising:
an instrument comprising:
a cartridge engagement unit, and
a manifold assembly; and
a cartridge configured to be removably engageable with the cartridge engagement unit and fluidically coupled with the manifold assembly, the cartridge comprising:
a substrate comprising a fluidic layer, a pneumatic layer, and a deformable layer between the fluidic layer and the pneumatic layer,
wherein the fluidic layer comprises:
a first side contacting the deformable layer and comprising a valve feature,
a second side opposite the first side,
a first fluidic channel,
a via in fluidic communication with the first fluidic channel and the valve feature, and
a second fluidic channel,
wherein the pneumatic layer is configured to transmit positive or negative pressure from the manifold assembly so as to control engagement between the deformable layer and the valve feature and thereby control fluidic communication between the via and the second fluidic channel in the fluidic layer.

25. The system of claim 24, wherein engagement of the cartridge with the cartridge engagement unit fluidically couples the cartridge with the manifold assembly.

26. The system of claim 25, wherein the pneumatic layer comprises at least one pneumatic channel configured to transmit pressurization fluid from the manifold assembly in the engaged state of the cartridge with the cartridge engagement unit.

27. The system of claim 24,
wherein the valve feature and the deformable layer form a diaphragm valve, and
wherein the pneumatic layer is configured to transmit the positive or negative pressure from the manifold assembly to selectively deform the deformable layer and thereby control engagement between the deformable layer and the valve feature so as to open or close the diaphragm valve.

28. The system of claim 24, wherein the instrument further comprises a thermal exchange device, wherein in an engaged state of the cartridge with the cartridge engagement unit, a portion of the cartridge is placed in thermal communication with the thermal exchange device.

29. The system of claim 28, wherein the portion of cartridge is a chamber and the thermal exchange device is controllable to thermally cycle the chamber through a plurality of temperature cycles.

30. The system of claim 24, wherein the instrument further comprises:
capillary electrophoresis module configured to receive fluid from the cartridge and perform capillary electrophoresis on the fluid.
